SEQUENCE LISTING (13 sequences)

SEQ ID NO: 1 - DNA, 230 bp, Homo sapiens
gtcaccaagcccgcggagaggaagcggctgtcactcggaagatgagaaattggagtctgagggaggctggcttctctagccctataagagggcaggcaccttttgaagcaaagaccagargagatggagctggacagagctgtgggggtcctgggcgctgccaccctgctgctctctttcctgggcatggcctgggctctccaggcggcagacacctgtccaggtaaggg

SEQ ID NO: 2 - DNA, 220 bp, Homo sapiens
acatggaggacacaccaggccaggcctcaggtataaagacttatactgtctgtaatgatgttactgcctgtaacgatgctcacatttcctcctgcacaggagattccctgaygttccacaacaaccagtccttctccaccaaagaccaggacaatgatcttaacaccggaaattgtgctgtgatgtttcagggagcttggtggtacaaaaactgccatgt

SEQ ID NO: 3 - DNA, 20 bp, Artificial sequence
  Synthetic
ctttgcccaaggccacaagc

SEQ ID NO: 4 - DNA, 19 bp, Artificial sequence
  Synthetic
ggaggccctggagtgccct

SEQ ID NO: 5 - DNA, 19 bp, Artificial sequence
  Synthetic
acatggaggacacacggcc

SEQ ID NO: 6 - DNA, 20 bp, Artificial sequence
  Synthetic
gctgccatgagtccccctga

SEQ ID NO: 7 - DNA, 20 bp, Artificial sequence
  Synthetic
aagaccagaagagatggagc

SEQ ID NO: 8 - DNA, 20 bp, Artificial sequence
  Synthetic
gctccatctcctctggtctt

SEQ ID NO: 9 - DNA, 20 bp, Artificial sequence
  Synthetic
attccctgacgttccacaac

SEQ ID NO: 10 - DNA, 20 bp, Artificial sequence
  Synthetic
gttgtggaacatcagggaat

SEQ ID NO: 11 - DNA, 15001 bp, Homo sapiens
tgtgttctatttattttacttaaatagtttatttaagcttgctcttttaagtgaaatttaacacccattcaccagagtgcataaactaaacctgcatcacccaatgaagccaaaacccatctccagccccccgtgccccttccaggcctaagggctccccctcctaagggagcggtcgtctcactggcccctgctaagggagcggtcatctcactggcatggtctgggcctttttctccacgttgccaccgagcatgcattccttcacactgctgttcagttttgcccactttcaaactctgtacagatgcaaatgaacagttcgcatgccttagtgctggcgccctggatgctccgtgtgcttgcggatttcaacaaagcttttgcgtgaagctggaatccgatcattttatgggtctacaggatgctccagtatgaacgaacctcagctgacggccccattctgctgcggatggacgtttgggttgtttccggcgtggggctggacattttcacacgtgtcttctttccagctgggcacacctctctgttgagtagaactgagctgaacatcgctggttatggaatatgctggtcgtagggaattgttgggaatactgccagttttccgaagtgcttgtaccaaggtttctgacacttcttcaccaaaaccttgcagagttggctgtttagaaaaattttagtgtctgcagtcatctcatgagatttaagttttcatttcttggattagttatgaagttgagcatgttttcatacttccatcatctattaaatattatcttttataagcgtttgttgaagtcactttcccactttctacaacatcgttgatttacagaaaccctttattctggatacaagccttttgtcacttctgtgtgttgaaacctctcttccctcaccctggctcgtcttttccctcccacggtggtgtattttgaaaaagaaataatactattttcatgatcaatttttccacttgtcctttatggtttgtggttttccatcttatttaaaataactgcaaggctattggagatcttataatatgatctaaaactgctgttttccctctcacatttgggtctacccttctcctgatgtggatttttttttcatctgataaaagtcaagtgtaacttcttgtttttccatatgactctccagtagtttgtgccactcattgaaagtttattctttcactattgctctttagagccaccatgtcataaatctagggtccgtatgtgcctggatttgcttctgcactccctactcacttccctaggtttgctgtgtacgcctaagccagccccacactgtctccatcactatcgcttgataatgactgctggtgtcacttcaagcaactcacccttgttttttcttgctcaagaatagcatggctatcagcctcttgcctttatataaattttatttatttatttatttatttgtttatttatttagacagagtttcactctgttgcccaggctggagtgcagtggtgcaatctcggctcactgcaacctccacctcccgggttcaagcaattctcccacctcagcctcctgagtagctgggactacaggcatgtaccaccacaccctgctaaatatatatattatatattttttgagatggaatctcactctgtcgtccaggctggagtacagtggctcgatcttggctcactgcaacctccgcctcctgggttcaagcaattctcctgtctcagcctcctaagtagctgggtttacaggtgcccaccatcatgcctagctaatttttgtatttttagtagagacagggttttaccatgttggccaggctggtcttgaattcctgacctcaagtaatctacctgtctcagcctcctaaagtgatgggattacaggtgtgagccactgcacccggccctttatataaattttaaattcaccatgtcaattgtcacagaaaatggtagtttaattgataatacagtggatatttagataaattaagagaagatgaatgtttttcctatatttggtattctaatccacacatatagaatatccttctaattattttaatcttcttttaggtctttctgttcctatttgatagttttgtacacagaagaccagcatggatgttgttagacttattcacagttatttgaatttggtgttgctgatgcaaattttttaaatttcatttttaaattctttgaggctagtttttagaagcacaattgatttttgctaaactttttattctgctagtttatctgtagaaactctagaattttctacatcctcaatcatgtcttctgaaaataacaactttgtgtctgcctttccaatctttttatggttttgttctcttgcctactgcactggacagtgacatgcaatacaatgttgaccacagtggtgatgacaagcatccttatttgttctcagtctctgaggatggttttccatttttcctgttaattatgttgtaacttctgtatccacagcttccaaagacctctttctatattctgttgtttctgttggtccttatacacattgtcttttattcccatgagcctggttatcgttgattgtgttttaacatttcatttgaaaaaaataaaacactttgcagaaataatgacctgggatgatgtcattctccttcagagaagatttttgtttctttcagcaaggagcctgggggcatgagcaataattccaggagttgagattcctttcctgggacacttagatgtctcaaaaccagtttgcagcccttgtgaatgttggttgttgctcactcatcctattgctagggtgcagcccttctgaaagccaaccccaaacccatacctttggcaggccgtaggccccagtctttgaccttcagctctgcaagatggtgaaatcactgcccaacttctcacctctcttttggagattggcaaatccctgtgcaaaaagaggtccccactactagtttcacttctctaagatttctgtggctcctgagagcttggcaaagtaatcctttactacatagctagcttgacggtgccttcgagcaaatctctttttaaaggttttgtctgtttgtccttaataggatgcccccaaatacctaaactgccattgccaggaatcaaatgacttttgttgatccaatgttaagatattttaacttgccagaatcttgcattctttcatactcttgggaacattgacttgatatctgaaggtggtgttgacttctcacctgaaataaacctattgatgttgcaaggaagaggaggtcccctcacacccagggggcagctggcaaatggaattgaggggcacagacctgctggctttctccctgttctgacccaatcccaggtgagtgctgctcctgaattggatcctctccaagaaaggaggccgcaccactgacacccagacctctcccgcctgggccagtagctcagggacactaagcggtgtctgaggcaatgaagcctcctgccgttgtctctcaacaggctggagccaacacacacaaacacacacagtcgagagtcgttgtggtgcccagagagcctggacacaaggtcctgtgtctcctggatcccccacccctgggccgcccctccacttctctcccacctggggagcccttgagtttggaaacccagtctacacacgctggctcagagaagttgcaaaccacaaaacaacacctctccctgctccttagtcctgctaccatctgactctcgttagtgactatttctatacgttctaaaaatcccgcacaaagtgtccacagcgtggcctggctaagcatgattcacagcacgagatgcggagtaggaacatggcctcagggatttgggggaacaagaggcatccccaccttcaggtgaaatgagcaatcccagtgatctacaaggcgttagacagaattgagtccaatgcctggctcttactagtggagtgacttcaatttctccagctgtaactttcaaataatttactccaccgaatggggctggtgcaagatgatatgatggaaatagtatttgggacataacaagtgctcaacatttttttcctctccatccatgataagtgcctgatgacggacaaaccctgaattttctgggaacagatgaggcaaggaagcgtcttcagcaaaaccttccttatcccccaaggttaaggtcaaggtttccgagtattgcttggggacagctggggaatggaaagggggtgtgggggctgtgttctccttaagccacaagcaaagccgatgctttccagcagaggagtctcaggagctctctggactgggagcagggtgtctttgctgggggaagagagcccaatggggtgggcatcttgctggagcctagtggaaggaaacatcctttgagatgacctcatacctcctgttggtgcaactaagcagcccataggaagtgggtgtcccccagggagttgagaaactgtggcacaaggcgagagctggtttcctctgccctgttagagctgggggactcttcagagtcaaaggccagagagcatggagctgagtggagccaccatggcccgggggctcgctgtcctgctagtcttgttcctgcatatcaagaacctgcctgcccaggctgcggacacatgtccaggtgagacagacctgggttttctggtccctaaaagctggtaacaaccccactcactgacactcgggtgtcccaggcaaaaccttttatgcacatcttcctgtgaagatgcccaacagatctgttttatcctcaggcaaggacatggaggctcagggcagctgaggcattgcccagggtcatggagggataggggagggatgtcaccaggtctgtctgcctcgaaagcccaggatttgcccattggagcttcataggtgggtctggaatctgtcactgagaaggaagaaaatactgttgactttatcttcttacgggcttaccagggaggcactctaagaccctctaatacggtggctgccacacggacttggggggtttgttaaaaaggcagatttctggctctcctccaagagagtcggaatcagttgatagggacaagggtttgaacggtggtaaggtgtttccctcactgattcaggagcaggtggttctgtgacgctcagcaagagactgctcctaaggacggcctggtgcccctggctgataacatatttatgaaggaaacataaatagtaaattacagcctgctctagtggaaggaggactggaaacacagaaaggaaataccccaaaaatggcagtgccatgtctccggtgagagcactgctccgttccatggtctctaagagcgcccttagcagggcgtgtagattttgtaatgaacaaaatgaaaaatattttttcaaagaagtaaacaactttggtttggctgcagctgtgccacacctggctctgtgtccttggaccccacactgcccctctccagaggacctagaaagccccttccagcccagtaattctacaaccacattcccaaggtctgggagggaaaatgctttgtcagccccaattccccaagtttcttagaaatattgattgtcattagttattgaaagtgcccgctgtttattaagctctttctaaatgccataaattacaataagagatttaaaatgcacattctttaaccaaatcctggccgcaaccttgttattatccccgcttggcagcccagggcacaaagctcagaggagttaagttacttgctgcaggacacacagcactgaggagatggggctcccctaggtcggggtggcctccaaagccagagctctgccccccatcacatctccgcctcaccagtgcctgaggggtgaggacagaccaaggccccagcagagcagaaaacctcacttcccagccactgttgggccaggcactgagtgggcacctgtgtttctctgcagaggtgaaggtggtgggcctggagggctctgacaagctcaccattctccgaggctgcccggggctgcccggggccccagggccaaagggagaggcaggtgtcattggagagagaggtaggtgcgggccgggtggggaggccaggcatggcaggggcactggctcttgctctttatgaaactggatgcagagttgggcaacacccccaccgtggttcctagatcaagagcctgggtcaggcccctgctagagccaggaacagagctgactcccagggcccaacagggttctgaaatggagggagggtctttcctaactatttcatgagctcacatttggacatgcttctgtagctctgtctcctagtccttgatccaataccagctttgctcagctactggcacgacttctaagctctgtgtccctccaccttctcacctgaaaattgaggtgaccgctttcttctgcacctgcctcatatgggaaaactgagactcaaagaggtcacaggctttcctacttgacaaaccagtaaggagtggggctgggacttggactggtctacttcagggcacagagggcacccgaatcctgagccctgagctctgcatgtggatttgcagggaacatgggagaatgaacagcaggcacctgccttgggtggttttgggtccccctaagcatggcataaggcagacgtgcacaaaaactcagaaagggcgctgccttagggggtgagctccccatgcctggacgcattcaagatgatgcctgcggagcacttggtggggatgtcccagtgtagatgggacaagctcaaatgtccctatgacttggactttcactgacgtgtctaggcttccgtgtgggtgtccggggccaggtcctctgtgcccactccgcaggctggacctgagagtgcggtcccgatcccctgctgagggaacactggggaggaagaccttccctggcaggagaagaatcaagtctggctcagacttcccttgaaccaggcttctaccagtgggcatggctggctaatataccctaaagcaagaaccagcccccatacactctccccagccactggggtcagcgagcgggctgggggctgcctcttggctggcatcggggacgggagggtcacctttctggccagaaccacgtggagcgctgtgtgacaccccaggagctcaggcgcacggtggaaccggatcctccctggcagcagcggctgcacgtggcgcccaccctctccgtgtggggacgtggtaggatgaggctctctgaggacgcattctttgttccagaacctggtcccttgctaggcagtagagctctgtgtgggtgacttgggtccaggcttatggtcaacagagtgtgaaacccaggttgctcccaaggcacaaacaggagcaaggtgcaccttgccttgagcccaaggtggggtgcaggcccctccctcaatccctcacctccatctgcagtcacccccaggctaaatccatggctagcaattctgagagctcctggaaccagaaggggttcaaggcagagtgggggagggtcaggacttaaccacttggggcaaagatttccagggagaattgtttaggttgcatttttctcagtgatatgtctttggaattttcaggagaacgcggtctccctggagcccctggaaaggcaggaccagtggggcccaaaggtaaccaggggacagacagcgaggaggccttgagcagggaccttggagctgtctgtaatgaaatgagggagggagagacagagacagagacacagacacagacacagagagacacggcaacagagagagaaagacagtggcaggcggacagggaccctgggatattccaggcctcggggcccctccccaccattgagacctgggcgttgtgatgccttagtgagcctgcagggctggggagatgcagtgtccaggccgggagcccactctgggaccagcctttccgtggcccacacatctggggtcaaaagctcattatcctgcctcttcctccaggagaccgaggagagaaggggatgcgtggagagaaaggtgagccacccgtgggcccaggcagttgtctctgcaggctttgtccaggggaggggtctcagaggcctcagtccgcctgcccaccgcccacgtccctgtcaatccaggctgtgggcagggaggggtggaaggagtccagcgggctttgttgtcagaatccacgagggctcaaaccctggtgtctacagcagtcactcccagtggccagcaggaccacatcagtgcatagatggtgacacttcaatatttcaatttctgtcttacttatgctcatttatgcaaaaattgtaaaagtttaaaaaagccaaatgtgcaagactctaatacactgtaccacgctccacccacaaacacaaatgaaaaccggagccgggggctgagggtccgccgatacctgagtcccaagaaacagaggccagggcggaggcagggcaggcttggagtgaccctgccaggcttgaagagttattccgctgttatcagtggcgaaggccccactaggggtccagttcgtccagcaggacccgcagcgcctgccacagcccaccaagctcaagctgcacagcaggggcctatggacaaccatgtggctggtgcctctggggaagtcgagtctggccatccttccctaggcctgtgccacgtgtcctggagacatcagaggagctgcctttgcccattctctattctgtgctcagggagccactggggctgaccaggacaaaggctctgatttcaagcttatgacaagtgtctcctctccccacacaggagacgctgggcagtctcagtcgtgtgcgacaggtgactgatcacaccctgacctcggtacccaagccttgcccccacccctggggccagctgcacacgttcacgctacggaatctaaggacagaaccctgtctgcacctatcatccccccctcaatcactcgagtaatgggcagctatcacctgaatcccttccatgacaacctgggctctactattatctaccctagaaggccgggtgcccaggaggcaaaggtggggcagacactgggaggtgggagggtcggcagactgggtgtgggcatcacagctgcgtggccctgggggccgtgtccctgtccaggcctcggagtcctgctcggtccgcacagaagcctgtggccctcctcctccctcctgggcctccctcccactgcccctgtcctgcccagggcccccgtcctgcagccattccccgggttcccttcccaggcccacgcaactgcaaggacctgctagaccgggggtatttcctgagcggctggcacaccatctacctgcccgactgccggcccctgactgtgctctgtgacatggacacggacggagggggctggaccgtgagtgtggggctgggcagaggcggtcagcctgggagtcccggaggccaggctgcacacctggttgtagaacacactctggaattctctattcccctggatggggacaatcatagatgatgggggaagtgacccgtgggtaaaagtccaggccatcggggaggctgggaggggaccggtgctctcctgctgtgtggcttggggcttgttgcctctcttctctgagaccgcatttcctctttcatcctgtggagtctgtgaggagaccagatgggcgtgcgtgtggcaccggcctgtgatgctctgccaactacaaatgctgctcctctggagggcgggtccccggtgctgtgggacctcggcctgccccgcctaactctgtatcctggcttcttcacaggttttccagcggaggatggatggctctgtggacttctatcgggactgggccgcatacaagcagggcttcggcagtcagctgggggagttctggctggggaatgacaacatccacgccctgactgcccagggtagggccactgccgtggctcaggggttggggggccctgaggtgctcctgccccttaggcctgggccatctgcaacataagcacctcttggcccacaggggattgggccctgagcacactcaggtggcactgggacctctaagggctgcgtcccctgccgcaggctctggcaacatcgggaagggaaacggttagagccctgggctgaaggcctcttcaggtcttggtcctactggcatcttagatcctgagagtgggaagcaggagagagaccactggggcttggggtagccttcccccatctccagctgttgatctgcctctgaagctggcatcttagtcccatggagagcccacgctcgcctcctccagcccacgcctcagcaggccccccacagccgcactcacctgagtgagagggcctccccccaaccctctggggcaggggatgagtttcacagctggtcctgaggcagtctgtataattgctggtccattatccctgcccactgccctgtggcatgattgaaggaatccactcaggtgcctcaaattcctgcaaaagaaactacaattagagcttcgcccactgctgtgttagtggctcaccaacccttagaaggagaaggtacactttcaaaatgaaagaatgctcccttccacccagtgaactccagtggggcatgttcagttctctgaaataacgggaaaggtgtttagaaacagaaagccatccctgagggcctgatagcaacaagggattttctgacatttccggttgatttttttcctggatgccacgttcagacatcccttaagaatatatccatccccagccgggcaccgtggctcacacctgtaatcccagcattttgggaggctgaggctggtggatcatgaggtgaagagatcgagaccatcctgaccaacatggtgaaaccccatctctactaaaaatacaaaaaaaaaaaaaagtagctgtgtgtggtggcacacacctgtaatcccagctactggggaggctgaggcaggagaattgcttgaacccaggaggcggaggttgcagtgagctgagatggtgccattgcactccagcctgggcaacaagagcggaactctgtctcaaaaaaaaaaaaaaaaaaattacccatccccagccctcatgcctggtgacagaaagatcttaaaggctggggccttaaaatctttttccaacaggaagcagcgagctccgtgtagacctggtggactttgagggcaaccaccagtttgctaagtacaaatcattcaaggtggctgacgaggcagagaagtacaagctggtactgggagcctttgtcgggggcagtgcgggtgagtgtctgcttggggctggtggcctgagtggggcttggaaagcagagagaacctggctctgtctcccctggtgaccttgggccctcctctctctgaatcccatttctccatctatgaagcagacactagcatttgtgtctctgtacagcgtggggctcacaggacatagcggctggtattcctaaggctatgtctgccgcacagtgagctgcctgggcagggctgagacgacccaccagcatcactgctgaaacctggaagacgtcgtccttttctcatgggtgccttggagctgtttcccaaactgtgctccatggaacaccagccttgtgggacattctttaccaaaaggagtctctggctacacgatctggggaggcatttcctacagggcgtctgcttagggaattcccagtgcacatcgttcgtgaaaggcactgagaattgctgcagctggaagtctgtgtaactaactgctccatggagcaaggcagtcccaggggagccttggaaatgacagccttggagtctgtgcagtcctttcacaggcacgatctcacgcatgacctccaaggaaggcagcagggcagggaaggtcacccccattttgcaggtggtaaaaccgaggctcagggggtgacatttctcagctataggagccagggatgagcttccggctcagggcttgagatagaggagaagcccccaggtgagggctgtcctgaacctaccaagggccaccaggggacgtggggagagaccggaaggagcggcgcaggctggaggagggaggtcattccaggagagagcaggaggcctgacagtttgcgggagccctggaggaaacaggtggttgctggagcaggggcctcaggttgtggagagagatttccagaccaggttgccagaggttgcctcctgcttccccttcctccaaggcctgggtgggtcatttgtgcacctgtagctcggctcatgcgtcgctaaagtgaggttggctctgctcagaggcaggatgtggtgagggtctcacagtggcagggctttttccagcatctgcaaaccccacattttctgtgagcaggtaattctctaacgggccacaacaacaacttcttctccaccaaagaccaagacaatgatgtgagttcttcgaattgtgctgagaagttccaaggagcctggtggtacgccgactgtcatgcttcaaacctcaatggtctctacctcatgggaccccatgagagctatgccaatggtatcaactggagtgcggcgaaggggtacaaatatagctacaaggtgtcagagatgaaggtgcggcccgcctagacgggccaggacccctccacatgcacctgctagtggggaggccacacccacaagcgctgcgtcgtggaagtcaccccatttccccagccagacacactcccatgacgcccacagctgcccctttgcccccagctcagtcaagccgccacatgcccacaacctcaccagagggagaattatgtttctaaatatgtttactttgggacagaaaagttttaccagtttcaatgtgtgtttcttgaacaccagtggttttgatcactttcctgtgtctgttgcacggcaggtctttccctggaatttggtaagtcgggaagagtcaggacccttatttagcagatggcactaccgatgttcagggaggttgagtgacttttgcgaggattttccacctccagtaattgacacagggcgatcctcacctccacccccaagcctcccccatccccccatgcctcactcacaacacttcacacttcttgaaaaaggtcactttggggtccagaaaacacttactaaaaagggggaaagtttagtaggaacttttctgcttcccaccacggcagagttcatgctttgggctggctggctgctgggaagagataaacacttagcactcactaagtgagcgcctggtcagccacctgcacagagaagattccgaagaggccttcctcccacacacagacctcagctagaggggaccgcggacctcagtgggaaaagctgcacaaggcttctaggagatgatggagaaggggcttctccagctcccaggccaggggagatttctcaaccacgacctaagaagcatcaaagggaatgtggcaagtgtgactccattcaaatcaagagctcccacttaccgaaggacactaagggaaccaaaggaaggccagaggggcagaatcaacgtgcggcacacaaatctttagttttctactattgccgcaaccagtcaccacaaatggagtggcttgaaacaacaagaactcgccacctcctggctcagtagatctggagtccctgggccttactgggccagcaggcaggactcagcagtggtggctccttccagggttctaggggagaacctgcttccttgccttttccagccccaggggtcactggcattcattggcccggactccttcccccaaccccaaagccagcagcaccacatctctctgacccttcatccgttgtcccctctccccccaagccccatgggacagggtctccactcctgagactcatgggattagactaggcctacctggatgatctgggatcctctcccatgtccaggtctctatccctaaccacatcagcaaagtctcttttgccatggaaggtatttgcaggctctgactttgtgtgtattagggcaggacatttttggcggggcagtggtgggggcagggaattattctgcctaccatgccttatacaaaaataaataaatcacaggcttatacctgaaagaattcctgcaaataataaaagaattagagcagctcagttaaataaaaaaaggtatgagatgtgaacagacacttcacagaagatgacatgcaggggagcaaggtatttatcaaaaagtgctccacttttcacttaccagagaaatgcaaatcagcacccaaaggagtcactgcggcaccctcatctgagcctcagaagttagaaagatggatagcaccagtgtcaaaggtgactgtgggagggagatttgcatacactgctagtaacaatgcgaattgatggaaccaacctggaaaatggcttggcatcaactgttggagaagaagacacacagtcctctaacctgccagtcccacatcagggcctggatatggccgtcatgtgtaccagggagcatggccaggaatctttacagccaaatactcaccatagcaccaaactggaatcagccgacatacccatcaggaagagaatggatgttaaaaaatgcaaggatagccattgggtagaacgccatacagccatgacaattacacagtgacacctacacacaaggagcatgagtctcacaaacatggcagcaaaaggagcctgttcaagagggtatgtgctatggattccattgtagataaagtttttcaaaagcaaaagtagccaatggtattttggatgcatatttaggtggtaaatgtatacaggaaaaataagaaagtgattgtatttgagggtgggaccacatggtgactgggaagggacagggacaagcctctggggttctggcaatgctctgtttcttgacccaggtggtgattgtaaggatgtttgttaactagtgatgaaaataatt

SEQ ID NO: 12 - DNA, 8700 bp, Homo sapiens
gggtcacagtttaaaatccttctactgatcttgccaaggaagaaggcacctcggcatcccgatggcagcaggtggcagggtgcggtggtggtagtgggaggtgcagtgctggttccgtctctgggggctttaggtatacggcctctggggccccacctgctactgtctctgtctctaacctggctcagtccaaggcgagagtgcaccttcaggccagctcttctccaaaaggcgaacacaaccactgaagtaagtccacagccatcctacaggtgggatgtctggcctcagggctcagggggttccctgggccctaggagtgaaattcacctactctcatcctccctacaggctaagcccctgcattcacactcaaggtctccccttcagatgtgtacctgggtcatagtcccccaggctccccactcttctctcctttccctcctgttcatgtgcccctgtgctctacatactgccccaggaaacagtggacacacacgcattttcctctccttttcaccctggcagcctgcctggacctcatagtggcagtttccaagcctcctgaggagagtcccaagtctgctcatgatgtggtgagggttactgtgtaaggcccacaccagtggcccatgaccagccctggccatgaggactctaggtattcggataagggagagagacctctttgaagcagaacatctttagcaaacccttccttgttccccgttctcagctcatggtgacatctgctcaccgctgaacaaaggtgggaggatgggcctgggctctttgcccaaggccacaagcaagtcagcctgttccctggaaggagaccgtccccctgcagggatgacagtcgccatgggatccccaaggggcggggggtagggagcagccctggagatgatctcgcacctcctgctggcgtcaccaagcccgcggagaggaagcggctgtcactcggaagatgagaaattggagtctgagggaggctggcttctctagccctataagagggcaggcaccttttgaagcaaagaccagaagagatggagctggacagagctgtgggggtcctgggcgctgccaccctgctgctctctttcctgggcatggcctgggctctccaggcggcagacacctgtccaggtaagggcactccagggcctcctcctggaaacttctcgtccctgaaagctggcagcttcgtgattggtggcttcctggcttccaggccttgcaccaggccgtgtggagcatcgtctaacgagacagcagctctgcatgcctcatcttatgtgaacaaacgcagaggccccgaatggatgtggcatctcagagacggagcttcctctgcttgccctcccataatggaaaccgaggcaggtttccagtctccctgggccacatgtccccctctaagcgtaaaaagcgtagtcatggtgttgtgtggggaggtgaggcttgtggtgccagctgggtagacaccagtcagatattctgccatggtcgccatcacagagagacagggtgtgaggtggggataagcacccggaaagacagggtgtcaggcggggatgagcactgcaggacctttccatccaggacctgctgggctctggagatggcagttcccatggtgtccctgatgcggcacaataagggtggcattgtccccgtgagttctggacctccttcggggctcccgaggggacactcgggtgccaaaccatgggctccggcgcatgcgagggcaggttgaaaatgcaggttctggagtcaggtcctgactccgtctacccggggctgggatgcagggtcaggaatttgtgatgctcctgatgcttgcagagattcgtgtcaggatttctggaatgcatgtgagacacagagctctgcggtgtgctgtgactagcatcgcattcaaggagcaatccgtggcgtgaagtgtgacacaggccaggaacagcggggattcgcttggaaactcctagtggctcgttgtggagtggtggggttagggtggcattattttcttccttaaatacttctagagtttctaaatgatcaaataatagcacgtgttatttttgtaacacactcacttgaatttgagttatctaattaaaaacaacaagctgcattccagctctagtttcgcaccaacttgttcagtgtccttgaaggacgcctccctctgtggaggaacaaggtggacccacaggagcccatccactctcagatctgctccaagatcaggcgtgggagttgcaatggtagcctctctccctgtgtccttctcacccgatattgattacaatgatatgatagatgagaacagacgagattaggatgctgtcttaaatggtaggagctgaaatgaacctgcacccctcggtaggtaagggaacagaggctcagaggggttaagggacttgtttcaggacacacagtactgaggaagtaaggtttgccttcaggcagggtagcctccaaggccagggttctgccccaaatcacctttctgcctgaccagtgcccaagaggtgaggacagaccaagtgcccaccagggcaggaaaacctcccttccaggtgaccctccagctgagggaggtggctgtggctatagccatctgcccagtcctgatgtcaccaagatggcagatgcctttcagttgagtggtatatctatggctcagtggagtcttcaggcccaggtgacactgagtggccacctgtgtttttctgcagaggtgaagatggtgggcctggagggctctgacaagctcaccattctccgaggctgtccggggctgcctggggcccctgggcccaagggagaggcaggcaccaatggaaagagaggtaggtgcaggcatggctgggggcactggctcttgctctttttgaaaccagatgctgagttgggcaacacccccaccatggttcctagatcgagagctgggtcaggcccctgcaagagccaggaaaagaactgactcccagggcccaacagggttctgacatggagggagcgtctttcttagctatttcatgagctcacatttggacatgcttctgtagctctgtctcctagtccttgatccaataccagctttgctcggccgctggcacgacttcaagctctgtgtccctccaccttcccacctcaacacggaggccatgacactcttctccacctgtctcagatgaggaaactgaggctcagagagtccagggatttcgctctgttctcacacttggaaggaaaagagccgagattctacccatggtctctggatttccagctcccggcccctgagtgaacaggagagccccgtgaggcctgggtcctggatctagaaccttctggtgcaggatgggcaagcctgggcggggccacagtcacgtcgtagcacgagcagggtcatggtcatgattggaaatgacagccgccagctccagggtgggccctttgatcctgggctgggcagttttcctcaagtcagtgtctttggaaattgcaggagaacgtggcccccctggacctcctgggaaggcaggaccacctgggcccaacggtaaggaggggacaagagtgagaagcggcttcaaggcccttccagacctggctgcagaggaacgtgaggcgggtcttctggggctgccacgctgtcctcgccagagccaacgcctgcccaggcagggctcttggggcttagtccagggcaggggtctcagtgtgggggaaggggtctcaatgtgtgtgtgggttctcagtctgggggagtggggttatcagtgtgggagctggtctcaatgtgtgtagggggtctcagtttgggggagtggggtcatcagtgtgggggcagggttctcaatgtcggaggaggggttcttggtggtgtagggagaggttcttggggtgtgtgttgggggaggggttcccggtgggcaggctgtggggaggtgttatcagcagggagggaggagctgtcaatatgggggagagattctgggatgggggagaggttctgattattcggggagggacttttagtgtggggaggggttcttagtgggggggctgtctgtatgggggggttctcaggtaggggaaggttttcagtgcaggagcatctgccctgtgggtgggcctgagaccaaatcctgtccctctaatgtggaagtggtcttgagagggtggggggggattgtctgcccacctctctaagcctcactcagtcctggcctctgtcccctgagcatgaggccctgtcttgtcccctcctcctgactctgcttgaaaaccaggatcagccccggatggcttggagaaccatgccaacaagttccaaacacagaattagtggaaattctctgctttaaaacaaagaattttctgatatattttcttgaggaataaattttgaggaatcggagaaatacgcagtaggaaattgaagctccctttactcactcacctttacccacagacctactcagaataagaccaccctacactcaccaaaccccaggctcagtcatgggttgtgtctaaacataaaatacgcaggtttggtgcccacaccgcaaacaacactccccacatcatattactgtgtccaccaagatgggctgtttgcaggccttggggcctcaggagaagcctgaagatccaggcttcctttagttggcatgtggaggcctcagcagtctctgggatggtggcctctgcttccccaggaacctctgaaaggggtgcatgtggtggaggggccagtgtaggcatccctcgagggaaagaaaaatttggtcaacagaaccagggtcagggactccttggctggacccataccatcacctcctggggaggcccagaaaatggtgtccgcggaccaatgggggctgaagggctctgatttccaaactgtgacacgtgtgtcctctctcatccatgaacaggagcacctggggagccccagccgtgcctgacaggtgactgaccacccccacactcctcccacggcttgtggctgcccttggctggaagtccagggtcatacgacgccattgccagaatgaagtgacaaatatgaacagaagaaaatggttgcttgcccgtttccttgtcccctaattattcaaggaagtgacaaatattgtccagacaacttacatacactccatggcaatttttttctatttataatctacgccagaaggccgggtgcccaggagctgaaggtgggggtgacactgggaggtgggagggccaggccaactggctgcggccatcacagctgcgtggccctgggggccgtgtccctgtccaggcctcagagtcccgctctgttcatacagacgcctatggccctgcttcttcctcccaggcctccctcctactgcctgtgccctgcccagggctcctgtcctgcagccattccccgggttcccttcccaggcccgcgtacctgcaaggacctgctagaccgagggcacttcctgagcggctggcacaccatctacctgcccgactgccggcccctgactgtgctctgtgacatggacacggacggagggggctggaccgtgagtgtggggctgggcagaggcggtcagcctgggagtcccggaggccaggctgcacacctggtgggagaacacactctggaattctctattctcctggtcggggacagtcagagatgatgggggagatgaccggtgggtaaaagtccaggcctttgggggaggctgggagaggttgggtgctctcctgctgtgtggcttggggcttgttgcctcccctctctgagcccccatttcctccttcatcctgtggagtctgtgaggagaacaggtgggcgtgctggtgaccccgcctgtgatgctctgccaactacaaatgctgctcctctggagggcgggtcccccgtgctgtgggacgtcggcctggcccccccggctcctgtcccctggcttctccacaggttttccagcggagggtggatggctctgtggacttctaccgggactgggccacgtacaagcagggcttcggcagtcggctgggggagttctggctggggaatgacaacatccacgccctgaccgcccagggtagggccgctgctggggcttgggggtcgggggccctgaatgggggtgcccctgcctcttgggcctgggctgcctggaacacaagagcctcttggcccacaggggattgggccctgagcacactcagggtggcactgggacctccgagggcttcgtccctgctgcaggctctggcagcatcgggacgggagagggtcagagctctgggatgaaggcctcttcaggtcttggccccactggcatctcaggtcctgagaggggaaggagggacacagtggccccatagtggagtttgggcccaaccccaactctgcctctaatccgctaggatttgggagtcaagagtcttgccctgctgctcagcacaccctgccgggtcagagctacacaggccccggggatgctgcggtgctctccgccctctgcctccatggagtccagacctccttccaggccctgccccagatccccagctcccatgtctaaaggtagagagcccttccgctgagaccctgaaacctttctctaacaggaaccagcgagctccgtgtagacctggtggactttgaggacaactaccagtttgctaagtacagatcattcaaggtggccgacgaggcggagaagtacaatctggtcctgggggccttcgtggagggcagtgcgggtgagtgtctgcttggggctgtgtggcctgggcttctgaggggggtttgggaagtggagagagcgtgctcagtgtcctggtagccttgtggaagaggcctcacctctctgagccaattcgtccatctctacatgcagacactaacatctgtgctctgtgtagcatgggggtcatgggacacatcagtgtatgactgcagaaatcccagcaagggcatctggtaggaaggaagtctctgcagagcagggacctatgcacggtgacagcagctgatatctctgcggggcatggctggaaggggtctgccatgatggaatccagagtagagaatgatgatcctgacccctgcctcctgttcttctgtgaaataaacatagtacacattaggtggcagggctagacctaaagacaatttgccaagaccactgccaacagcagggcagtattcactggagtcatggattgcacttcttggattgtgcagtgcacaacctgcctaaccatacatggaggacacaccaggccaggcctcaggtataaagacttatactgtctgtaatgatgttactgcctgtaacgatgctcacatttcctcctgcacaggagattccctgacgttccacaacaaccagtccttctccaccaaagaccaggacaatgatcttaacaccggaaattgtgctgtgatgtttcagggagcttggtggtacaaaaactgccatgtgtcaaacctgaatggtcgctacctcagggggactcatggcagctttgcaaatggcatcaactggaagtcggggaaaggatacaattatagctacaaggtgtcagagatgaaggtgcgacctgcctagcccaggccggcctcagggtcaggacgcctccacacatagttggttggggggtagggttgggagcttggccctacggtttgtaaaagaaacacatgtcgtgattctaaattgggtttgtcttgctgtgcggaagaggttcaaatccagctctgtttccgcaatcaaccctttcagatttgtgacattaactcacctcaaacctctgtctttccttgacaatgagtaagaatgtgttcctgaccatgccagcaggaaccttccttaaggacaaatacaagactttgaagctgcgatgggctcagagtgcagccctgctcaggtccctgggcaggctaaacgccttcctggggctcgaccatctgggcttctctgtgagctggatggcagggaagtcagaaggcacattggagaggaagtttctcttgcactcagagatgccgttgcctgtccaaggcccccagcctcgtagccgcagctctgctcactctcccccgtcacccaggcttcctgccgcctgctgtcctgggtattcgtgggatctgctcgctacttcctcacaagccgaagcaagagtggcagtgtgcctgtgactccctcatcaaaacggagagagtggttacatgaggcaggtgtgagggacccgagcactcctgaggagggaagaacactcctcttggccatgagtgtccatcccaaagctgccctgcagcctagcccagtgcagattgaatcagaaagccaaagccccttccacccgcttctctcaggcaagctgcctgcaagagagaaaagtgtattagtgcaggagcttgtgtctgctgaggatggcacctggccctctcctctcaccacgtccgggtcctcctgaggaccggtcccctgtccccggtccccagccagctcagcctgaccaggcttttagaggctccgcaccccagcatcggccagtgctgacccttgtacggctgtttgggtccccgatcactctcataacagccctgcccttgtcctataccctctactcagagaggatccagaagggtccaaggaggcc

SEQ ID NO: 13 - DNA, 10001 bp, Homo sapiens
ggaaagggccccagggactccacgcagccattcctggccttcctctctcaggctctcctgggaaggcctgccttctgtccccaggcaccgaagtcttccctgacggtaacatcgcccatggatgggcgcttgccaggagccaggcatgtagctgcgagttttataactttttattttattttattttttcttttcttttcttttttttttttcttttttttttttgagacagagtctcactgtgtcgcccaggctagagtagagtggtgagatcttggctcactgcaacatccacccccccctgccaaccacagtccacctcctcacccctcccttgcaccttcacttccgccaaagctccaagcctctggaaatcagcctcccctttgtgcactcccccgtctccctagagcccctgctgcttcccctgtatgggatatgataacaccccatctgcccttgccttctctcttctctaggcttctgcatgagcagctcctcctgcctgggtcaccctgcccttgtttctcccccagcatgagttttcttcttcaccctcctcctttattgggtgctttctttttttgcaaaagtagtaaatgcctgtgagggtttgaaaaaggactatacagaagagtgagaactgggctgggtgcggtggctcatcccagcactttgggaggccgaggcaggaggattgcttgagctcaggagttggagtcagcctagtcagcctgggcagcatggcgaaaccccatctctactaaaaataccaaaattagctgggtgtcgtggtgcacgcctgtagtcccaagtactcaggaggctgaggtgggaggatcacttgagcctgagaggtccaggctgcagtgagccaagatcatgccactgcactccagcctgggtgacagagtgagaccctgtatatattaaaaaataaaaaactggccaggaggtatagtggctcacacctgtaattccagcactttgggagactgaggcgggatgattgtttgaagccaggagtttgagaccagcctgggcagtatagtgagaacccatctctacaaaaaataaaaatttaggcggggcaaagtggctcacacctgtaatcccagcactttgggaggccaaggcgggtggatcacttgaggtcaggagtttgagaccagccagaccaacatagtgaaaccctatctctactaaaaatacaaaaattagctgggcgtggtggcacacacctgtaatcccagctacttgggagactgagtcaggagaatcacttgaatccaggaggcggaagttgcagtgagccgagatcaagtcactgcactccagcctgggcaacacagcaagactttgtctcaaaataaataaataaaataaaaatttagcccggtgtggtggctctgcctggcatcccagctaccgggagactgaggtgggaggactgcttgagcccaggaggtcggggctgcagtgagctatgatcacaccgccacactctagcctaggtgagagagtgagaccctgtctccaaaaaaaaaaaaaaaaaaagagagaacaactccttccctggtcctactctccagaaataaccacagttcacaaggtctgtgtcagacctctttctgtgaatttacaaaatacatatatacacacctctagcattttaaaaatacaaagtgggataatgctgtgcatactgttttgcatttttgcccctctcattttagatcagattcaccctgtgagttgtctgaatggtcctaattcatgtgaatgccatcaatgactggttgggtcatttccacactgatgcttacaaacagcgccatggtaaacaggcttgtctgcagatcttggcatactcgtgggacagtctggggaagacagattcctagaactggaattgcggtctcaggatatccctctcgcggacacatcctgtcaggcaaacactgcctccacttcccctgataccacttgcacctcagccctctccataagcaccagggaggtgcagaggaggccttgagggagggtttcctgaatgagtgaatgaatgagccctgcctctcctcccttatcacctgaggtccctctccagctcaggcctgatccttcacatctggaccctggctgctgcgtcctccctggcttcccggcctccaggccttcctacatgggcttcccgctcaattggcgtacattagcagctgtttcctaagcctcacgggactcaaggttgagcatggagtcactttctttttttttgagacggagtttttgctctttcgcccaggctggagtgcagtggtgcgatctcggctcattgcaacctctgccttcgggtttcaagtgattctcttgcctcagcctcctgagtagctgggattacaggtgcatgccaccaagcccggctaatttttgtatttttagtagagacagggtttcaccgtgttagccaggctggtctcgaacccctgacctcaggtgatctgcctgcctcggactcccaaaatcccgggattacaggcatgagccaccgcagccagccagaatcactttcttaatggtgcttttccctggcgggggcatggggcttgggtaaaactctgttgctctcagcatataagtggccaagagaacagaagtcacgtcccatcactgggcctcagtttctctttccttattcatttaactctttcttactgagcacccattttgtaccaggcccataatgcaataataaggattaatagcaagtccatgtgaggatcggaggtgacgtcagtatgacgctggaatgcagtagatgcccaggaaatggtagcagttgtcactattcatagcattgacaaggatcagggaaggattcccagtggagatggctgtagagctagaccttcatggacagatggggcttcacaggtagagacgactgtgaatgtagaggtctggagagatggctggggagccctggaagccaggatcagaagttggactctttcagtgtgcggtggctcacacctgtaatcctagcactttgggaggccgaggtgggtggatcacctaaggtcaggatttcgagaccagcctggccaacatggtgaaaccccttctatactaaaaaaaaaaatacaaaaaattagctgggcttggtggcgtgcacccataatcccagctacttgggaggctgaggcaggagaatcacttgaacccgggaggcagaggttgcattggttgcattgagctgagagcacgccattgcattccagcctgggcaacaagagtgaaactccgtctcaaaaaaacaaaaaaacaaaaaacaaaaaacaaaaagatgttggactccatcctggttggatagggcagggataatgccctttctctctggggctgaaggaggaaatactcccaccccctgctggctccagtctgggtggggtgtccctgggggacaatgggaggaagtcttgtttcatgtcctcccaccagcctggcccccttataatctggcatccttcccctggtgggcccagcaagatggatctactgtggatcctgccctccctgtggcttctcctgcttggggggcctgcctgcctgaagacccaggaacaccccagctgcccaggtgggcacctagggtgccctcctcaccaacccactcctcattccctcgctgggtgtccccccaccaaggctcatcaacctcaaggagcagggtgggagtttcataatctgggctctgcatgtaaatatatgcaaatggatgcaaagcacacgaaaccttgccagctcaggagacaagcatcctgacagtgggaatctgggagacaggaagagaactgaagaggggtctggaagagacagtctaccctgtgcccaccctgcctgtgtctccctgtaggacccagggaactggaagccagcaaagttgtcctcctgcccagttgtcccggagctccaggaagtcctggggagaagggagccccaggtcctcaaggtgagaggggctggggagcgggctgggatcttgggaatggggaaggcaggaaagtagcaatttttgtctgtgctgaggggccacgacaaaggagcaaggaacctactctgtgccagctccaggctgggcacccggcatcctaagatcaactctgccaggtaagtggtttcatccccattctgctgaggaggaaagggaggttcagagaggataagatatttgcccgcaatcacaaagtgagtgaagggtgaaactgggatttgaacctctggctccaagtctcttgtttctttttggtcctgggcatggggtgggactcaggaaatctctgcctttgtgatttcagggccacctggaccaccaggcaagatgggccccaagggtgagccaggtgagtaagtggggctggaggcctccccgctttctttgcctctgtccctggactctagggtgggatctctgcttggctgctgtgcctatgatggctctctcacaggggaatggatggctgggaggagtgggaaggattaggagaagggcctggctaccctatgatggccctcctattggggatggagggctgggaggagtggggaggattaggagaagggcctgggtaccctatgaaggccctcctacagggtttggaaggctgggaggagtggagaggattaggagaagggcctgggtaccctatgatggccctcctacagggtatggaaggctgggaggagtgggaaggattcggaaaagggcctgggtaccggctccactggtggctcagcgctggggtgctgagacatcagagccaacactcatcccttctctggggcaggagatccagtgaacctgctccggtgccaggaaggtgaggcagcccttgcaggagccaggaacttgggatcacatggtgttgggtgttggcagacccccaagtgtggccaagatcctccccacagcctcctcagaacccttcttcagggacctgaaggagccaagagggtccctagggtcccacacttgttccagtgcctgggggtgggggtcagggaataacctgggcaccaccccagtccatctcccctttcccaggccccagaaactgccgggagctgttgagccagggcgccaccttgagcggctggtaccatctgtgcctacctgagggcagggccctcccagtcttttgtgacatggacaccgaggggggcggctggctggtgagtgtctgagaaatgggctaacaggccaggcccactggtcacttctcccccttggcccctaggggagccagaacccaccagagcaaggcatttcctagaggaattcactgacaaatgtttagtgagcccccaatgggacctgaagaagtatgagggcttcacagaggaggtgccttttgaaaagaccttaagttgagtgcagtagctcacgcctgtaatcccagctacttgggaggctgaggcaagagaattgcctgaacccaggaggtggaggttgcagtgagccgagttcttgccattgcactccagcctgggtgaaaagagtgaaactctgtctcaaaaaaaaaaaaaaaaaaaaatagggtagggaggggcattccagctagaagtaaagataaactcagggaggcagcagagtaaaaggtttgcagggaacgcgtgagtgagctgagagcaggcgtgggtgtcggctaaagtctgccggcactgacggaacatggattgaggacctctgtaaaggccagttcatggaaggcactaaatgatcaacttaagaagtgtgcagcgcgcccagacgcggtggttggctggacgtggtggctcacgcctataatcccagcactttgggaggccaaggcgggcagatcacctgaggtcaagagttcgagatcagcctggccaacatggtgaaaccccatctttactaaaaatataaaaattagccaggcgtgatggtgggcgcctgtaatccaagttattcaggaggctgagctgggagaattgcttgaaccccggaggtggtggttgcagtgagccaagattgagtcactgcactccagcctgggcaacggagtgagactctgtcttaaaaaaaaaaaaaaaaaagaagagtgtgcagggcaatgggtagccccaggagagttttaagcaggggaggggccaagtagaacttctgttttaaaaagatgattcgggctacaccatgaaagatgcactgcattgggggaccagtggaggcagggacatggtttaggaagacagcaaggagccaggcagtaaagacagtggagcaggcctgacacagtagctcacacctgtaatcccagcactttgggaggccgaggtgggtggattgcctgagctcaggagttcaagaccagcctgggcaacatggcaaaaccccatctctactaaaaatacaaaaaattagctgggcgtggtggcaggtgcctgtaatcccagctatgcagaaagctaaggcacgagaattgcttgaaccagggaggcagaggttgcagtgagccgagatcacaccactgtactccagcctggtaacagggggtgagactctatctcaaaaaaataaataaataaaataagacagtggagcaatggaaatagaagtacttaggttgacaactctataggacctggggcctacttggcttggagggggaggtggaagacatcagggtaaggccaggggtccagcttgggctgtgggagtggcctctctaatgtaaaatatcttggtttttttgtttgtttgtttgttttgagacggagtcttgctctgctgcccaggctggagtgcagtggtgtgatctcggctcactgcaacctccacctcccaggttcaagcagttcttgtgcctcagcctcctgagtagctggaactacaggtgtgagccaccacacccggctaatatttgtatttttagtagagacgggatttcaccatgttggccaggctggtttcaaactcctgacctcaagtgatccacccgcctcggcctcccaaagtgctggaattacaggcatgagccaccaagcgttcttggttcttatagtaaatgaaattgggttattttgactgacacccagcagcgctggccatctcctgtgggtggggacagtgttttgtcaattcagtcttcccagggcttgcaaggctgtggcatgaagttgagtgtcagtaaaagttaacaagggaatgtagagttcatagaaatattcaaagctaacttgaaggatccccattttatcaacaggccccaatccaaagagagagtgggtatccacccacccctcacagttcctgcaccctcagctgccagtgcccagcctggagtaatagctgctgtgccccccctcccccaggtgtttcagaggcgccaggatggttctgtggatttcttccgctcttggtcctcctacagagcaggttttgggaaccaagagtctgaattctggctgggaaatgagaatttgcaccagcttactctccagggtgagttcccagcagggtcctggagtctgggggttactgtctgccatcctgaatccctgccccacccccaagaactgaggattcctccatccctgcctccccttctttccaggtaactgggagctgcgggtagagctggaagactttaatggtaaccgtactttcgcccactatgcgaccttccgcctcctcggtgaggtagaccactaccagctggcactgggcaagttctcagagggcactgcaggtgagtgagcctaaggggagcagaggaaggaggctggatcctttcccagtgctgccactgcccccatgaatgacccagggcaatcctgtctcctctgtgggccttagtttccgcatctgagaaatggcaatcatgaaacaggatggtgcaacagtgaagaatttgggatctggaggcaaacagacctgagtttgaatccatctctgccatttagtacttatgtgaccttgggcaagtcctacccctccactgagcctcagtttcctcctctataaaatgggcagggaggccgggcacggtggctcatgcctgtaaccctagcactctgggaggccgaggcgggcagataacttgaggtcaggagttcaaacccaacctggccaacatggtgaaaccccgtctctactaaaaatacaaaaaaattagccgggcgtggtggagggtgcctgtaatcccagctactcgggaggctgaggcaggagaatcacttaaatctgggaggcagaggttgcagcgagccaagagagtgtcactgcactctagcctgggcgacagagcgagactccatctcagaaaaaaaaaaaaaggcggggggagcgcagggaggcaaggaggaagttttgggtgccgcctgaggctctgttgagtgttagtgttctctgctttggaagaggaaccacggaagtcttctttcagagaaacagtgactattggggagagatttaaaagagggaagtggttatgagggagtgcctcagggaaccacctttcctggccaggcacctaagaggttcctcccagcttttcctagcagagttgctcagactggatgggagctgagagaagacaaaggagctagaggggggccggggattctggagggggtaagacagggggaaaagctgggagctttggagccagacagtcccgagttcaagtcctgccgctgcttcaaccagccgagtaaccctgcgcaaacaacctggccccttggtgttgctcgtggggcctgataaaattcaactcttagttgctctgagactcccatggcattatatctccaaaggtgccaggcgtggtgctgggcaccaaggtgctttccatcccttccctgctaggggattccctgagcctccacagtgggaggccctttaccacctatgacgctgaccacgattcaagcaacagcaactgtgcagtgattgtccacggtgcctggtggtatgcatcctgttaccgatcaaatctcaatggtcgctatgcagtgtctgaggctgccgcccacaaatatggcattgactgggcctcaggccgtggtgtgggccacccctaccgcagggttcggatgatgcttcgatagggcactctggcagccagtgcccttatctctcctgtacagcttccggatcgtcagccaccttgcctttgccaaccacctctgcttgcctgtccacatttaaaaataaaatcattttagccctttcaagactcttccagttactgggaaaaaaccctagagggcagagcagggacacaagtttcagctccattcagggaagaatttccttccagtcaggaagatggaaaggacagcctctaaagtagtgaggcttctgtctctggaggtgtgcaagacaaggccagaggggcatatagtggaaggactggaggagattcaagcatccattgacaatttcctgaggctctctgggtcctgcaagtggatggctttgtggcaggaattgcacaattgcacagcttttttgtttgtttgtttgtttgtttgtttttgagacgcagtctcgctctgtcgcccaggctggagtgcagtggcaccatcctggctcactgcaagctctgcctcccaggttcacgccattctcctgcctcagcctcccgagtagctggaactacaggcacccgttaccacgcccggcgaattttttgtatttttagtagagacggggtttcatcatgttagccaggatggtctcaatctcctgacctcatgatctgccctcctcggcctcccaaagtgctgggattacaggtgtgagccactgcgcccggccgctttttttttttttttttttttgagatggagtcttgctctgtcgccctgcccaggctggagtgcaatgagccagaggggcatacagtggaaggattagaggagatccaagcatccattgacaatttcccgaggccctctgggtcctgcaagtggat

(19) United States
(12) Patent Application Publication
(10) Pub. No.: US 2008/0131888 A1
(43) Pub. Date: Jun. 5, 2008

(54) Nucleic Acid Variants in the Ficolin Genes Associated with Altered Innate Immunity

(76) Inventor: Lieve Nmi Nuytinck, Drongen (BE)

Correspondence Address: HOWREY LLP, C/O IP DOCKETING DEPARTMENT, 2941 FAIRVIEW PARK DRIVE, SUITE 200, FALLS CHURCH, VA, 22042-7195, US

(21) Appl. No.: 11/795,532

(22) PCT Filed: Jan. 20, 2006
(86) PCT No.: PCT/EP06/50342

§ 371 Date: Jan. 7, 2008

Related U.S. Application Data
(60) Provisional application No. 60/649,195, filed on Feb. 2, 2005.
(60) Provisional application No. 60/714,272, filed on Sep. 6, 2005.

(30) Foreign Application Priority Data
Jan. 21, 2005 (EP) 05100395.2
Aug. 30, 2005 (EP) 05107936.6

(51) Int. Cl. C12Q 1/68; C12Q 1/00; C07H 21/04

(52) U.S. Cl. 435/6; 435/4; 536/22.1; 536/24.31; 536/24.33

(57) ABSTRACT

The present invention relates to a method and kit for identifying a subject at risk of, or having, an indication associated with altered innate immunity. The present invention is based on the determination of ficolin genotypes and/or serum levels.

### FIELD OF THE INVENTION

The present invention relates to a method and kit for identifying a subject at risk of, or having, an indication associated with altered innate immunity. The present invention is based on the determination of ficolin genotypes and/or serum levels.

### BACKGROUND ART

Immunity to infection is mediated by two systems, the acquired (or adaptive) immune system and the innate (or natural) immune system.

The innate immunity system is an evolutionary ancient form of immunity and offers the main resistance to microbial pathogens within the first minutes, hours or days of an infection (Fujita et al., 2002).

Innate immunity recognition is mediated by germ-line-encoded receptors, which means that the specificity of each receptor is genetically predetermined. The strategy of the innate immune response may not be to recognize every possible antigen, but rather to focus on a few, highly conserved structures present in large groups of microorganisms. These structures are referred to as pathogen-associated molecular patterns (PAMPs), and the receptors of the innate immune system that evolved to recognize them are called pattern-recognition receptors (PRR). PAMPs can be protein, lipid, nucleic acid, and carbohydrate (Lu et al., 2002).

As soon as the PRRs identify the corresponding predetermined carbohydrate pattern on a pathogen, they immediately trigger effector cells to destroy the invading microorganism, rather than after having to undergo a proliferative cycle, as is the case for the time-delayed adaptive immune response. PRRs can be divided into three classes: signaling, endocytic, and secreted (Medzhitov R. et al., 2000).

The complement system is an important arm of innate immunity. The complement cascade can be activated through three distinct pathways, i.e. the classical, the alternative and the lectin-pathway. The lectin pathway involves carbohydrate recognition by PRR, such as mannose binding lectin (MBL) and ficolins, and the subsequent activation of associated enzymes that are known as MBL-associated proteins (mannose binding protein serine protease or MASP), MASP-1, MASP-2 and MASP-3, and its truncated form, small MBL-associated protein (sMAP, also called Map19) (Matsushita et al., 2000; Fujita T., 2002; Lu et al., 2002).

The collectins belong to the Ca2+-dependent (C-type) lectin superfamily characterized by the presence of the C-type carbohydrate recognition domain (CRD). The lectins possess a different type of lectin domain, called the fibrinogen-like domain. The collectin family has five well-characterized members: MBL, lung surfactant protein A and D, bovine conglutinin and collectin-43. The ficolin family consists of three members: H-ficolin, L-ficolin and M-ficolin (Lu et al., 2002). Binding of collectins and ficolins to microbes through the lectin domains leads to activation of multiple immunological processes such as complement activation and phagocytosis.

Ficolins, like MBL, are lectins that contain a collagen-like domain. Unlike MBL however, they have a fibrinogen-like domain, which is similar to fibrinogen α- and γ-chains (Matsushita et al., 2000). Ficolins also form oligomers of structural subunits, each of which is composed of three identical 35 kDa polypeptides. Each subunit is composed of an amino-terminal, cysteine-rich region, a collagen-like domain that consists of tandem repeats of Gly-Xaa-Yaa triplet sequences (where Xaa and Yaa represent any amino acid), a neck region, and a fibrinogen-like domain. The oligomers of ficolins comprise two or more subunits, especially a tetrameric form of ficolin has been observed.

Some of the ficolins trigger the activation of the complement system substantially in similar way as done by MBL. The fibrinogen-like domain of several lectins has a similar function to the CRD of C type lectins including MBL, and hereby function as pattern—recognition receptors to discriminate pathogens from self.

In human serum, two types of ficolin, known as L-ficolin (P35, ficolin L, ficolin 2 or hucolin) and H-ficolin (Hakata antigen, ficolin 3 or thermolabile b2-macroglycoprotein), have been identified, and both of them have lectin activity. L-ficolin recognises GlcNAc and H-ficolin recognises GalNAc. Another ficolin known as M-ficolin (P35-related protein, Ficolin 1 or Ficolin A) is not considered to be a serum protein and is found in leucocytes and in the lungs. L-ficolin and H-ficolin activate the lectin-complement pathway in association with MASPs. Absolute ficolin deficiency states have not yet been described, although low levels in chemotherapy patients compared with healthy controls have been observed (Minchinton R. M. et al., 2004). Kilpatrick D C et al. (2003) did not find a significant relationship between plasma ficolin concentrations and chemotherapy-related infections. Atkinson A P M et al. (2004a) identified low L-ficolin concentrations in children with recurrent respiratory infections. Hummelshoj T et al. (2003), identified genetic polymorphisms in human ficolin genes, i.e. 7 polymorphisms in FCN1, 11 in FCN2 and 1 in FCN3. Thus far, no genetic association studies with ficolins have been reported.

Individuals vary considerably in their susceptibility to infection and in their ability to recover from apparently similar infectious processes. These differences can be partially explained by polymorphisms of the genes encoding proteins involved in mediating and controlling the innate immune response, the inflammatory cascade, coagulation, and fibrinolysis. Molecular biology has revolutionized medicine by increasing our understanding of the pathophysiological mechanisms of disease and the ability to assess genetic risk. Recent data suggest an association between specific genotypes and the risk of adverse clinical outcomes. Continued identification of such allotypes and haplotypes may not only provide insight as to why the response to treatment varies amongst individuals, but also may potentially decrease morbidity and mortality through improved risk assessment and the administration of prophylactic or “personalized” medicine.

Despite the different associations of genetic risk factors with certain diseases, there is a continuous search toward more accurate genetic and/or biochemical markers that provide a reliable diagnosis or prediction of the risk to develop a disease or disorder influenced by innate immunity.

### SUMMARY OF THE INVENTION

The present invention provides a method and kit for identifying a subject at risk of, or having, an indication associated with altered innate immunity, based on the ficolin genotype, concentration or functionality.

In a first embodiment, the present invention provides a method and kit of identifying a subject at risk of, or having, an indication associated with altered innate immunity, comprising detecting in a sample the presence or absence of at least one nucleic acid variant in at least one ficolin gene.

More particular, the present invention provides a method and kit of identifying a subject at risk of, or having, an indication associated with altered innate immunity, comprising detecting in a sample the presence or absence of at least one nucleic acid variant in at least one gene, or part thereof, selected from the group consisting of:

the FCN1 gene,
the FCN2 gene, and
the FCN3 gene,
whereby the presence of at least one nucleic acid variant identifies whether a subject is at risk or has an indication associated with an altered innate immunity.

Specific regions of interest in the ficolin genes are:
the promotor region and/or exon 9 of the FCN1 gene,
the promotor region, the 5′UTR region, and/or exon 8 of the FCN2 gene, and/or
the IVS5 region of the FCN3 gene.

The methods and kits of the present invention can also be carried out in combination with other methods for identifying a subject at risk of, or having, an indication associated with altered innate immunity. In a preferred embodiment the methods and kits are carried out in combination with a method for the detection of the presence or absence of a nucleic acid variant, or other markers, in any other gene.

Any detection method is part of the present invention. Preferred methods and means for the detection of the presence or absence of the nucleic acid variants of the present invention are hybridization, sequencing, PCR, primer extension, MPLA, OLA and restriction site analysis.

In a further embodiment, the method and kits of the present invention identify a subject at risk of, or having, an indication associated with altered innate immunity, and comprises measuring the concentration or functionality of at least one ficolin protein in a biological sample, wherein an increased or decreased ficolin concentration or altered functionality compared to a reference value representing a known health status indicates that said subject is at risk or suffers from a disorder associated with altered innate immunity.

A further embodiment of the present invention relates to a method for selecting an appropriate treatment or therapeutic agent for a subject at risk of, or having, an indication associated with altered innate immunity, comprising determining the presence of an indication associated with altered innate immunity by the methods of the present invention and selecting an appropriate treatment or therapeutic agent.

The present invention also relates to a method for detecting the presence or absence of a nucleic acid variant in the FCN2 gene comprising determining the nucleotide at position −4 and/or 6359 in the FCN2 gene, and thereby detecting the presence or absence of the nucleic acid variant. Also nucleic acids, kits, primers and probes for use thereof are covered by the current invention.

### FIGURE LEGENDS

FIG. 1: Genomic organisation of the FCN1 gene located to chromosome 9q34.

FIG. 2: Genomic organisation of the FCN2 gene located to chromosome 9q34. Four different splice variants are also given.

FIG. 3: Genomic organisation of the FCN3 gene located to chromosome 1p35. Two different splice variants are also given

FIG. 4: Flanking sequence of two FCN2 polymorphisms in the 5′UTR region and in exon 8.

FIG. 5: human FCN1 wt gDNA sequence (SEQ ID NO:11): exon sequences and possible polymorphism positions are indicated in respectively grey and bold/boxed.

Nucleotide +1 is the A of the ATG-translation initiation codon which is bold and underlined.

Further specifications are:

| | | | |
|---|---|---|---|
| LOCUS | NT_019501 15001 bp DNA | PRI | 16-OCT-2001 |

DEFINITION Homo sapiens chromosome 9 working draft sequence
segment.
ACCESSION NT_019501 REGION: complement(1334080..1349080)
VERSION NT_019501.6 GI:16160566
COMPLETENESS: not full length.
FEATURES Location/Qualifiers

| mRNA | | complement(join(2125..2580,3668..3802,5021..5150, |
|---|---|---|
| | | 5551..5678,6116..6148,6920..6955,7292..7345,8884..8997, |
| | | 10305..10499)) |
| | | /gene=“FCN1” |
| | | /product=“ficolin (collagen/fibrinogen |
| | | domain-containing) 1” |
| | | /transcript_id=“NM_002003” |
| | | /db_xref=“LocusID:2219” |
| | | /db_xref=“MIM:601252” |
| | gene | complement(2125..10499) |
| | | /gene=“FCN1” |
| | | /note=“FCNM; Located on Accession NT_019501” |
| | | /db_xref=“LocusID:2219” |
| | | /db_xref=“MIM:601252” |
| | CDS | complement(join(2333..2580,3668..3802,5021..5150, |

5551..5678,6116..6148,6920..6955,7292..7345,8884..8997,
10305..10407))
/gene=“FCN1”
/note=“Located on Accession NT_019501”
/codon_start=1
/protein_id=“NP_001994”

FIG. 6: human FCN2 wt gDNA sequence (SEQ ID NO:12): exon sequences of transcript SV0 (Transcr 2; NM_{—}004108) and possible polymorphism positions are indicated in respectively grey and bold/boxed. Nucleotide +1 is the A of the ATG-translation initiation codon which is bold and underlined.

Further specifications are:

| | |
|---|---|
| LOCUS | NT_019501 1852354 bp DNA linear CON 20-AUG-2004 |

DEFINITION Homo sapiens chromosome 9 genomic contig.
ACCESSION NT_019501
VERSION NT_019501.12 GI:29731701

| gene | 488123..494831 |
|---|---|
| | /gene=“FCN2” |
| | /note=“Derived by automated computational analysis using gene prediction |
| | method: BestRefseq. Supporting evidence includes similarity to: 4 mRNAs” |
| | /db_xref=“GeneID:2220” |
| | /db_xref=“MIM:601624” |

mRNA(Transcr 1)join(488123..488232,489837..489950,490613..490666,

492051..492083,492550..493208,493741..493875, 494479..494831)
/gene=“FCN2”
/product=“ficolin (collagen/fibrinogen domain containing lectin) 2 (hucolin)”
/note=“unclassified transcription discrepancy; Derived by automated
computational analysis using gene prediction method: BestRefseq. Supporting
evidence includes similarity to: 1 mRNA”
/transcript_id=“NM_015838.1”
/db_xref=“GI:8051587”
/db_xref=“GeneID:2220”
/db_xref=“MIM:601624”

mRNA(Transcr 2)join(488123..488232,489837..489950,490613..490666,

492051..492083,492550..492677,493079..493208,
493741..493875,494479..494831)
/gene=“FCN2”
/product=“ficolin (collagen/fibrinogen domain containing lectin) 2 (hucolin)”
/note=“unclassified transcription discrepancy; Derived by automated
computational analysis using gene prediction
method: BestRefseq. Supporting evidence includes similarity to: 1 mRNA”
/transcript_id=“NM_004108.1”
/db_xref=“GI:4758347”
/db_xref=“GeneID:2220”
/db_xref=“MIM:601624”

mRNA(Transcr 3)join(488123..488232,490613..490666,492051..492083,

492550..492677,493079..493208,493741..493875, 494479..494831)
/gene=“FCN2”
/product=“ficolin (collagen/fibrinogen domain containing lectin) 2 (hucolin)”
/note=“unclassified transcription discrepancy; Derived by automated
computational analysis using gene prediction
method: BestRefseq. Supporting evidence includes similarity to: 1 mRNA”
/transcript_id=“NM_015837.1”
/db_xref=“GI:8051585”
/db_xref=“GeneID:2220”
/db_xref=“MIM:601624”

mRNA(Transcr 4)join(488123..488232,489837..489950,490613..490666,

492051..493208,493741..493875)
/gene=“FCN2”
/product=“ficolin (collagen/fibrinogen domain containing lectin) 2 (hucolin)”
/note=“unclassified transcription discrepancy; Derived by automated
computational analysis using gene prediction
method: BestRefseq. Supporting evidence includes similarity to: 1 mRNA”
/transcript_id=“NM_015839.1”
/db_xref=“GI:8051589”
/db_xref=“GeneID:2220”
/db_xref=“MIM:601624”

CDS(Transcr 1) join(488133..488232,489837..489950,490613..490666,

492051..492083,492550..492677,493079..493208,
493741..493875,494479..494726)
/gene=“FCN2”
/note=“isoform a precursor is encoded by transcript variant SV0; ficolin
(collagen/fibrinogen domain-containing lectin) 2; ficolin (collagen/fibrinogen
domain-containing lectin) 2 (hucolin);
/exception=“unclassified translation discrepancy”/product=“ficolin 2 isoform a
precursor”
/protein_id=“NP_004099.1”
/db_xref=“GI:4758348”
/db_xref=“GeneID:2220”
/db_xref=“MIM:601624”

CDS (Transcr 2)join(488133..488232,490613..490666,492051..492083,

492550..492677,493079..493208,493741..493875, 494479..494726)
/gene=“FCN2”
/note=“isoform b precursor is encoded by transcript variant SV1; ficolin
(collagen/fibrinogen domain-containing lectin) 2; ficolin (collagen/fibrinogen
domain-containing lectin) 2 (hucolin);
/product=“ficolin 2 isoform b precursor”
/protein_id=“NP_056652.1”
/db_xref=“GI:8051586”
/db_xref=“GeneID:2220”
/db_xref=“MIM:601624”

CDS (Transcr 3)join(488133..488232,489837..489950,490613..490666,

492051..492083,492550..492797)
/gene=“FCN2”
/note=“isoform c precursor is encoded by transcript variant SV2; ficolin
(collagen/fibrinogen domain-containing lectin) 2; ficolin (collagen/fibrinogen
domain-containing lectin) 2 (hucolin);
/product=“ficolin 2 isoform c precursor”
/protein_id=“NP_056653.1”

/db_xref=“GI:8051588”
/db_xref=“GeneID:2220”
/db_xref=“MIM:601624”

CDS (Transcr 4) join(488133..488232,489837..489950,490613..490666,

492051..492091)
/gene=“FCN2”
/note=“isoform d precursor is encoded by transcript variant SV3; ficolin
collagen/fibrinogen domain-containing lectin) 2; ficolin
(collagen/fibrinogen domain-containing lectin) 2 (hucolin);
/codon_start=1/exception=“unclassified translation discrepancy”
/product=“ficolin 2 isoform d precursor”
/protein_id=“NP_056654.1”
/db_xref=“GI:8051590”
/db_xref=“GeneID:2220”
/db_xref=“MIM:601624”

FIG. 7: human FCN3 wt gDNA sequence (SEQ ID NO:13): exon sequences of Transcript Variant 1 (NM_{—}003665) and possible polymorphism positions are indicated in respectively grey and bold/boxed. Nucleotide +1 is the A of the ATG-translation initiation codon which is bold and underlined.

Further specifications are:

| | | |
|---|---|---|
| LOCUS | NT_037485 | 10001 bp DNA linear CON 19-AUG-2004 |

| DEFINITION Homo sapiens chromosome 1 genomic contig. | |
|---|---|
| ACCESSION NT_037485 REGION: 1806067..1816067 | |
| VERSION NT_037485.3 GI:29791382 | |
| FEATURES | Location/Qualifiers |

| | |
|---|---|
| gene | complement(746..6458) |

/gene=“FCN3”
/note=“Derived by automated computational analysis using
gene prediction method: BestRefseq. Supporting evidence
includes similarity to: 2 mRNAs”
/db_xref=“GeneID:8547”
/db_xref=“MIM:604973”

mRNA(transr 2) complement(join(746..1111,2230..2364,2477..2606,

4770..4897,5599..5643,5990..6085,6362..6458))
/gene=“FCN3”
/product=“ficolin (collagen/fibrinogen domain containing)3 (Hakata
antigen)”
/note=“unclassified transcription discrepancy; Derived by
automated computational analysis using gene prediction
method: BestRefseq. Supporting evidence includes similarity to: 1
mRNA”
/transcript_id=“NM_173452.1”
/db_xref=“GI:27754777”
/db_xref=“GeneID:8547”
/db_xref=“MIM:604973”

mRNA (transcr 1)complement(join(746..1111,2230..2364,2477..2606,

4770..4897,5122..5154,5599..5643,5990..6085,6362..6458))
/gene=“FCN3”
/product=“ficolin (collagen/fibrinogen domain containing)3 (Hakata
antigen)”
/note=“unclassified transcription discrepancy; Derived by
automated computational analysis using gene prediction
method: BestRefseq. Supporting evidence includes similarity to: 1
mRNA”
/transcript_id=“NM_003665.2”
/db_xref=“GI:27754775”
/db_xref=“GeneID:8547”
/db_xref=“MIM:604973”

CDS (transr 2) complement(join(870..1111,2230..2364,2477..2606,
4770..4897,5599..5643,5990..6085,6362..6452))

/gene=“FCN3”
/note=“isoform 2 precursor is encoded by transcript
variant 2; ficolin-3; collagen/fibrinogen
domain-containing lectin 3 p35; collagen/fibrinogen
domain-containing protein 3; Hakata antigen; H-ficolin;
ficolin (collagen/fibrinogen domain-containing) 3 (Hakata antigen);
/codon_start=1/product=“ficolin 3 isoform 2 precursor”
/protein_id=“NP_775628.1”
/db_xref=“GI:27754778”
/db_xref=“GeneID:8547”
/db_xref=“MIM:604973”

CDS (transr 1) complement(join(870..1111,2230..2364,2477..2606,

4770..4897,5122..5154,5599..5643,5990..6085,6362..6452))
/gene=“FCN3”
/note=“ficolin/opsonin p35 lectin family
isoform 1 precursor is encoded by transcript variant 1;
ficolin-3; collagen/fibrinogen domain-containing lectin 3
p35; collagen/fibrinogen domain-containing protein 3;
Hakata antigen; H-ficolin; ficolin (collagen/fibrinogen domain-
containing) 3 (Hakata antigen);
/codon_start=1
/product=“ficolin 3 isoform 1 precursor”/protein_id=“NP_003656.2”
/db_xref=“GI:27754776”
/db_xref=“GeneID:8547”
/db_xref=“MIM:604973”

FIG. 8: Wild type frequencies of four SNPs in the FCN2 gene are compared between a group of individuals with low L-ficolin levels in serum versus a group with high L-ficolin levels as measured by the method described by Atkinson et al. 2004b

### DETAILED DESCRIPTION OF THE INVENTION

Unless defined otherwise, all technical and scientific terms used herein have the same meaning as commonly understood by one of ordinary skill in the art. All publications mentioned herein are incorporated by reference. The materials, methods and examples are only illustrative and not limiting.

According to the present invention, the determination of the nucleic acid sequences and/or the ficolin concentration or functionality makes it possible to estimate or identify if a subject is at risk of, or has, an indication associated with altered innate immunity.

In a first embodiment, the method of the present invention determines the presence of both variant and normal nucleic acids of one or more of the ficolin genes in a sample. As used herein the term “ficolin gene(s)” refers to the gene ficolin 1 (M-ficolin; FCN1), ficolin 2 (L-ficolin; FCN2) and ficolin 3 (H-ficolin; FCN3), and also to analogous, variants or derivatives thereof.

More specific, the current invention relates to a method of identifying a subject at risk of, or having, an indication associated with altered innate immunity, comprising detecting the presence or absence of at least one nucleic acid variant in at least one gene selected from the group consisting of:
the FCN1 gene,
the FCN2 gene, and
the FCN3 gene,
   whereby the presence of at least one nucleic acid variant identifies whether a subject is at risk of, or has, an indication associated with an altered innate immunity.

The term “nucleic acid” refers to a single stranded or double stranded nucleic acid sequence and may consist of deoxyribonucleotides or ribonucleotides, nucleotide analogues or modified nucleotides, or may have been adapted for therapeutic purposes. There is no limitation in length. A nucleic acid that is up to about 100 nucleotides in length, is often also referred to as an oligonucleotide.

The reference nucleic acid and protein sequences indicated in the current invention are derived from GeneBank (NCBI) and indicated by their respective accession number, as is well known to the person skilled in the art. The nomenclature for the ficolin nucleotide and amino acid changes as used herein is generally accepted and recommended by den Dunnen and Antonarakis (2000). Frequent updates of the nomenclature for the description of sequence variations are provided on the web-site of the Human Genome Variation Society.

Accordingly, the nucleotide numbering of the coding DNA and RNA reference sequence is as follows:
nucleotide +1 is the A of the ATG-translation initiation codon
there is no nucleotide 0
the nucleotide 5′ of the ATG-translation initiation codon is −1.

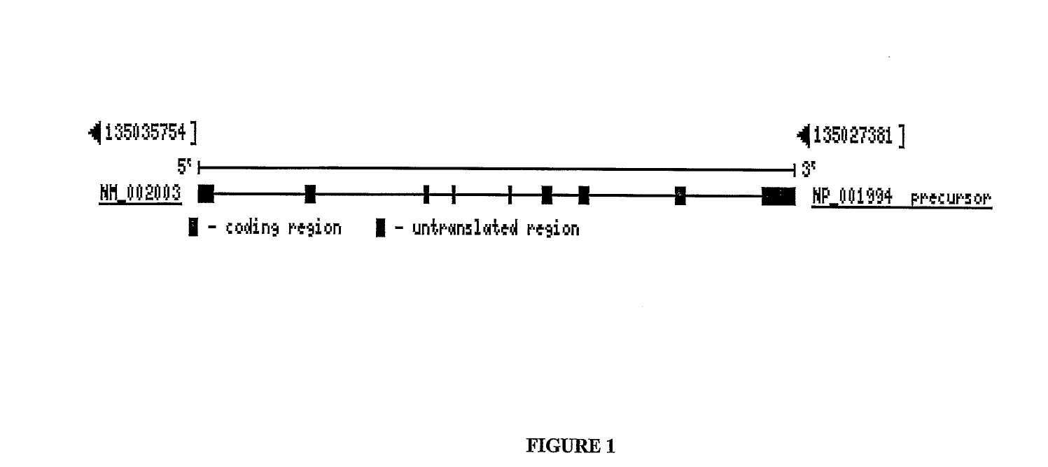

For ficolin 1 (FIG. 1), also indicated as FCN1, the reference nucleic acid sequence is NT_{—}019501 (gDNA; Version: NT_{—}019501.6, GI:16160566; SEQ ID NO:11) and NM_{—}002003 (mRNA). The reference protein sequence is NP_{—}001994.

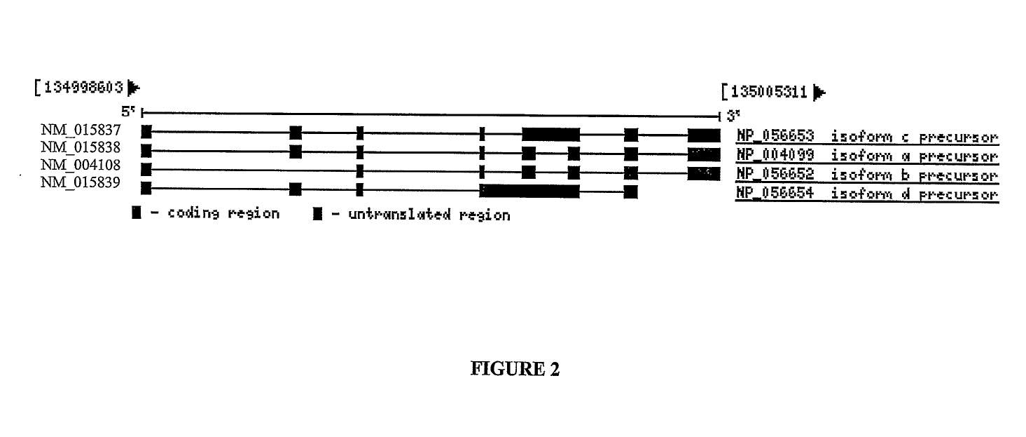

For ficolin 2 (FIG. 2), also indicated as FCN2, the reference nucleic acid sequence is NT_{—}019501 (gDNA; Version: NT_{—}019501.12, GI:29731701; SEQ ID NO:12).

Four different transcripts of the FCN2 gene arising from alternative splicing, and encoding different isoforms of ficolin 2, have been described.

The splice variant SV0 is the most predominant FCN2 gene transcript in the liver and encodes a protein of 313 amino acids. It represents the longest ficolin 2, isoform a.

The reference mRNA sequence is NM_{—}015838, and the protein encoded thereby is NP_{—}004099.

The splice variant SV1 is a minor FCN2 gene transcript in the liver, and results from the deletion of exon 2. Since the reading frame is unchanged by this splicing event, it encodes a shorter protein of 275 amino acids. The reference mRNA sequence is NM_{—}004108, and the protein encoded thereby is NP_{—}056652.

The splice variant SV2 is a minor FCN2 gene transcript in the liver, and is generated by the persistence of the fifth intron between exons 5 and 6. It encodes the same 143 amino acids in the amino-terminal end as the SV0 transcript variant, plus an additional 39 amino acids from the intron sequence. The reference mRNA sequence is NM_{—}015837, and the protein encoded thereby is NP_{—}056653.

The splice variant SV3 is a minor FCN2 gene transcript in the liver, and is generated by the persistence of introns 4 and 5. Exons 1-4 of this transcript encode a truncated protein of 102 amino acids, followed by an in-frame stop codon at the beginning of intron 4. The reference mRNA sequence is NM_{—}015839, and the protein encoded thereby is NP_{—}056654.

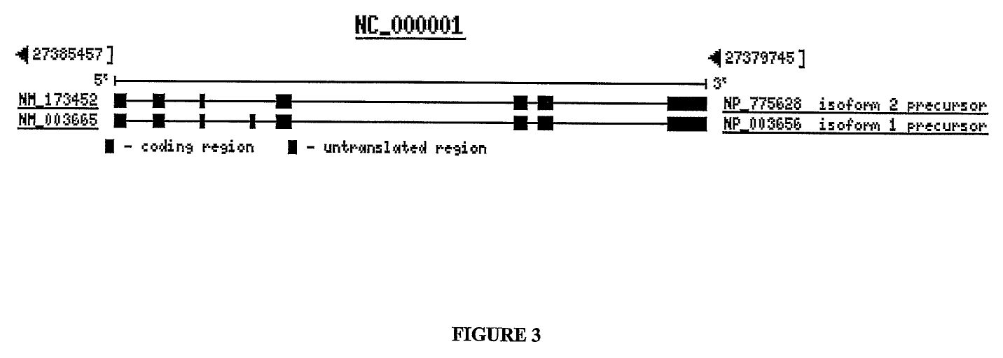

For ficolin 3 (FIG. 3), also indicated as FCN3, the reference nucleic acid sequence is NT_{—}037485 (gDNA; Version: NT_{—}037485.3 GI:29791382; SEQ ID NO:13).

Alternative splicing occurs at this locus and two variants, each encoding a distinct isoform, have been identified. Transcript Variant 1 represents the longer transcript and encodes the longer isoform 1. The reference mRNA sequence is NM_{—}003665. The protein encoded thereby is NP_{—}003656.

Transcript Variant 2 lacks an alternate in-frame exon, compared to variant 1, resulting in a shorter protein (isoform 2) that lacks an internal segment compared to isoform 1. The reference mRNA sequence is NM_{—}173452 and the protein encoded thereby is NP_{—}775628.

The term “nucleic acid variant” or “polymorphism” or “variant” as used in the present invention, means that the nucleic acid sequence at a certain position in the ficolin gene differs relative to one or more reference nucleic acid sequences.

The most simple nucleic acid polymorphism is a polymorphism affecting a single nucleotide, i.e. a single nucleotide polymorphism or SNP. Nucleic acid polymorphisms further include any number of contiguous and/or non-contiguous differences in the primary nucleotide sequence of the nucleic acid under investigation relative to the primary nucleotide sequence of one or more reference nucleic acids. The term “polymorphic position” or “position” refers to the nucleic acid position at which a nucleic acid polymorphism arises. Nucleic acid sequences comprising at least one such polymorphism are referred to as “polymorphic nucleic acid sequences”, “polymorphic polynucleotides”, “polymorphic sequences” or the like. The polymorphism or nucleic acid variant can be an insertion, deletion, substitution, tandem repeat or similar.

The term “haplotype” means a particular pattern of sequential polymorphisms found on a single chromosome. As used herein, the term “allele” is one of several alternative forms of a gene or DNA sequence at a specific chromosomal location (locus). At each autosomal locus an individual possesses two alleles, one inherited from the father and one from the mother. The term “genotype” means the genetic constitution of an individual, either overall or at a specific locus.

In a specific embodiment, the present invention relates to a method according to the present invention, wherein the ficolin genotype has at least one variant allele of the ficolin gene (heterozygous). In a further embodiment, the method of the invention relates to a method according to the present invention, wherein the ficolin genotype has two variant alleles of the ficolin gene (homozygous).

The invention relates in particular to any polymorphism located within at least one of the FCN genes as identified in SEQ ID NO:11, 12 and 13, or in the corresponding cDNA or RNA sequence. Preferably, the polymorphism is located in the promotor region, at least one of the intron regions (IVS) and/or at least one of exon regions of the FCN gene.

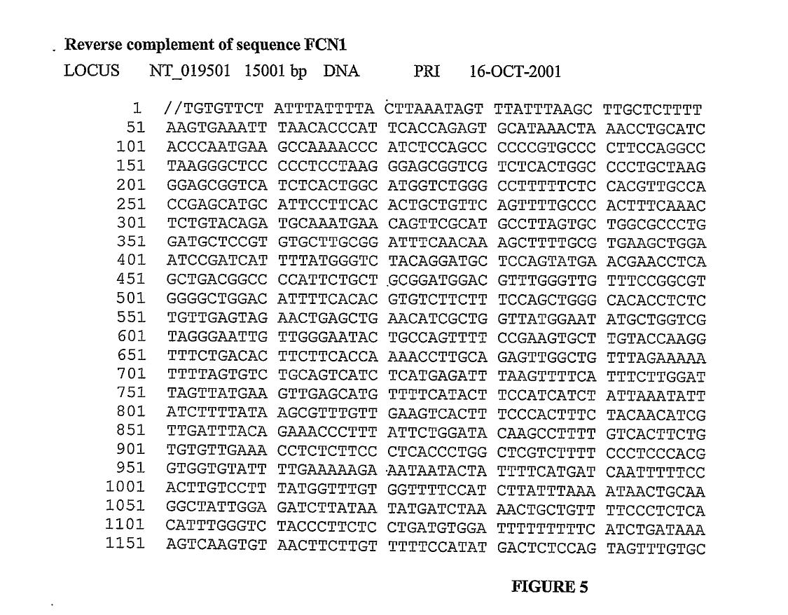

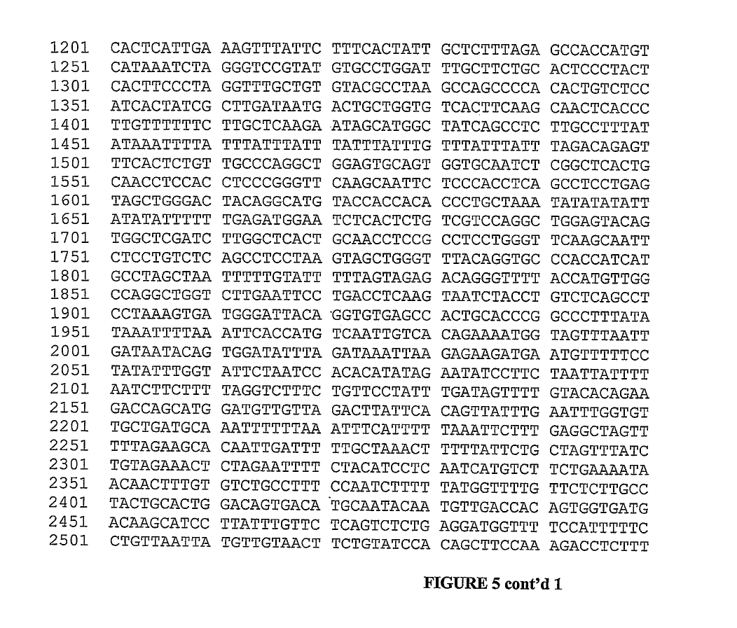

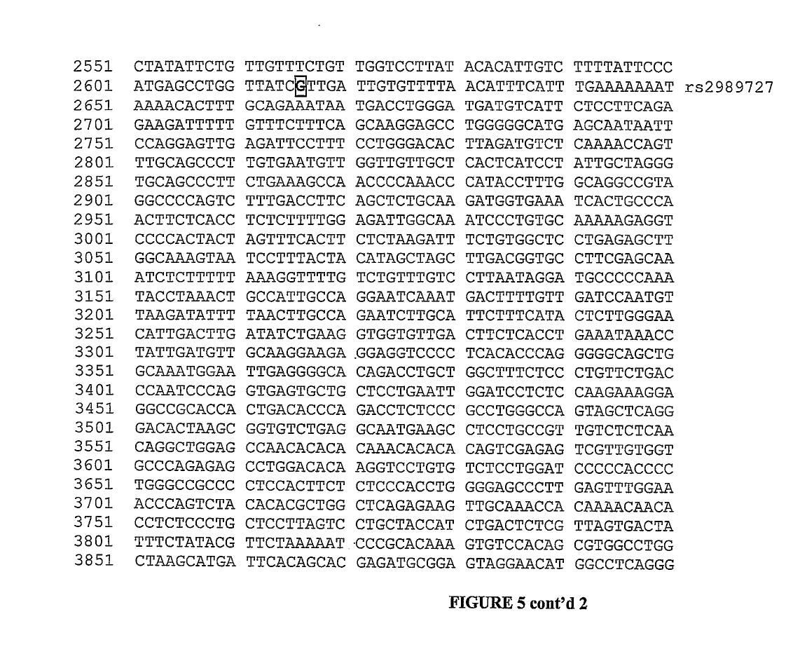

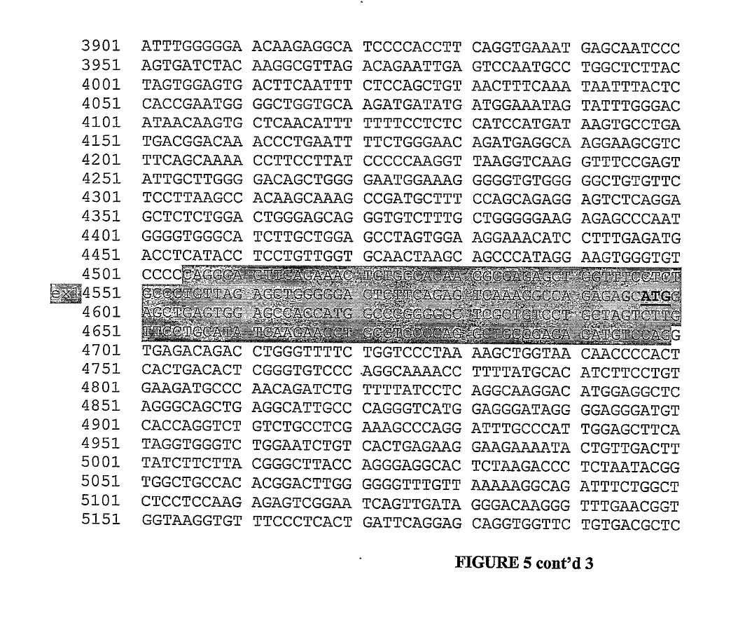

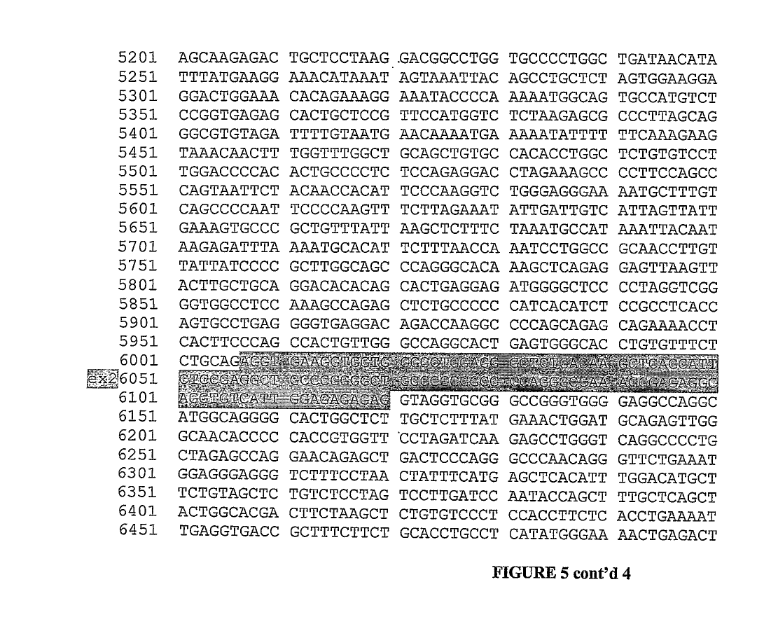

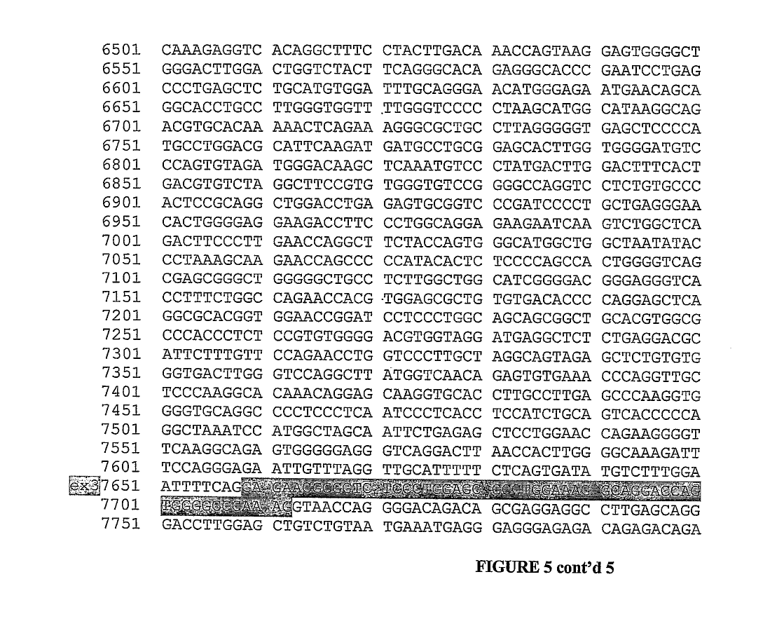

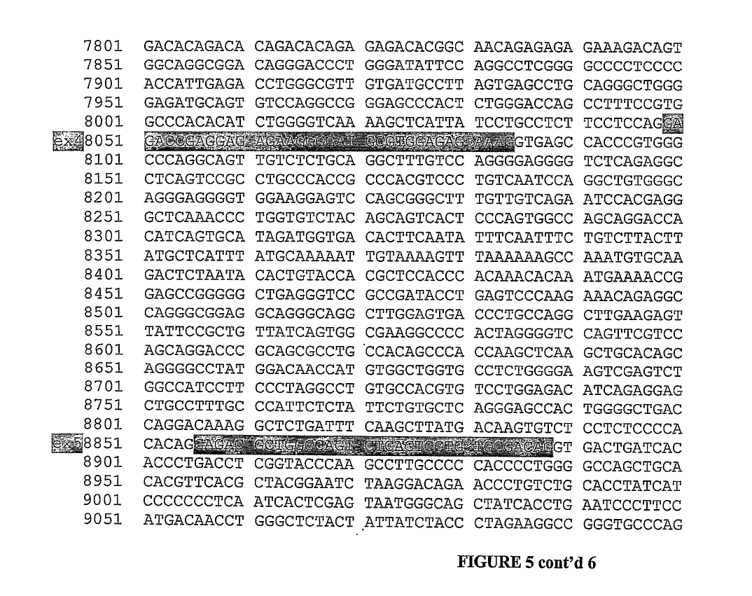

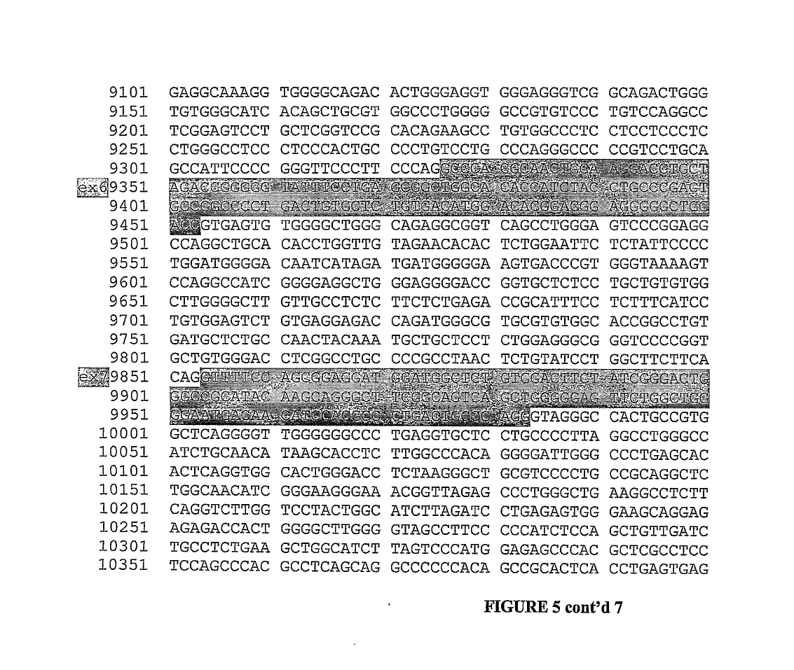

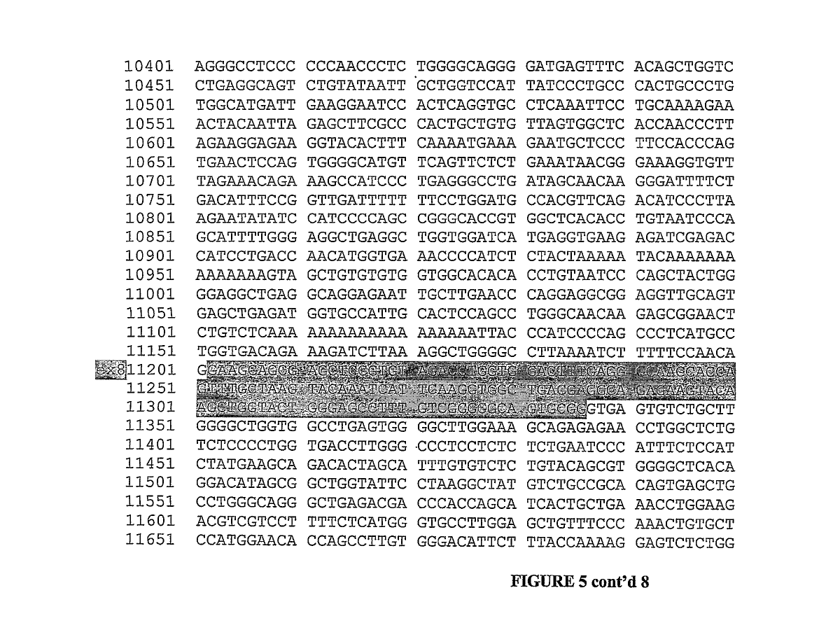

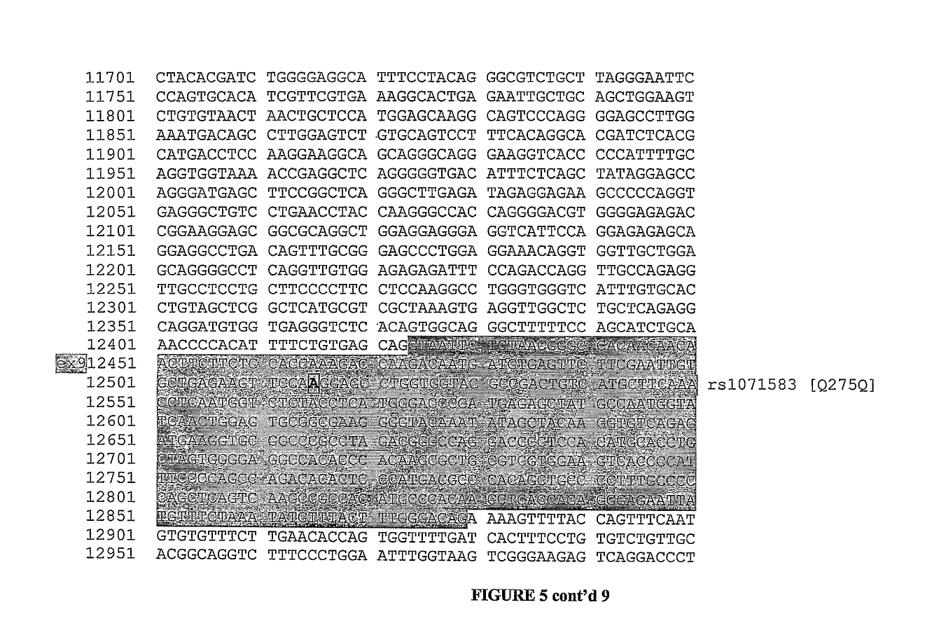

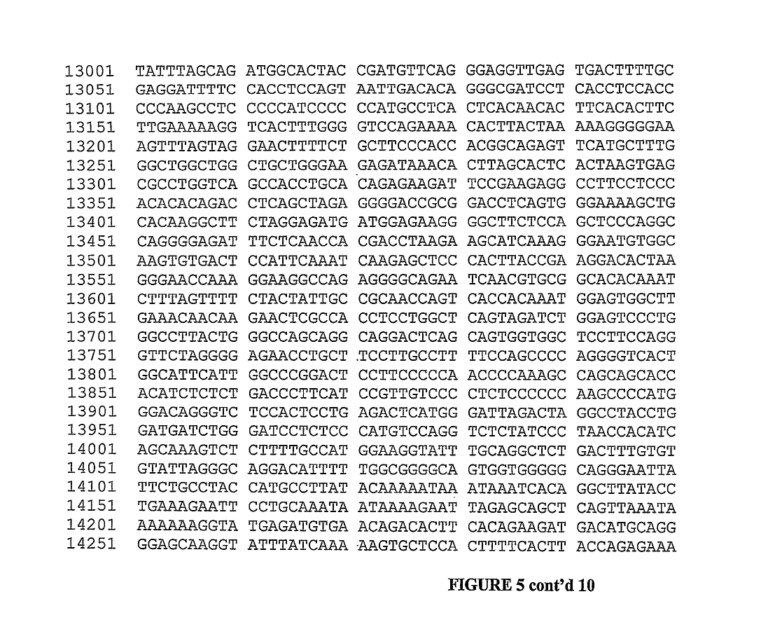

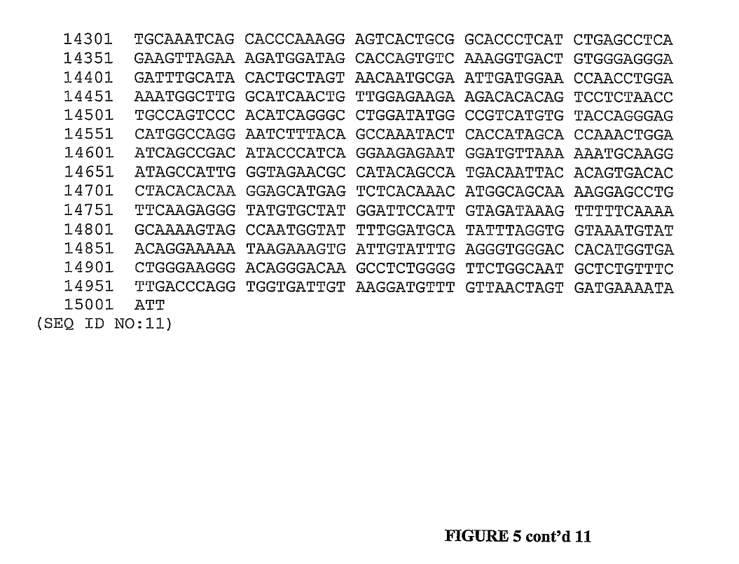

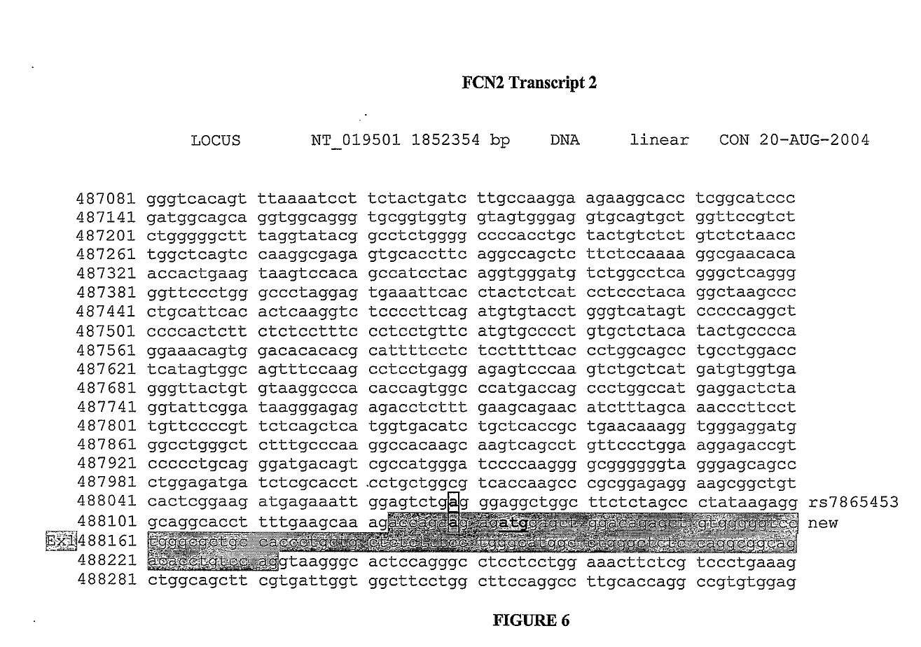

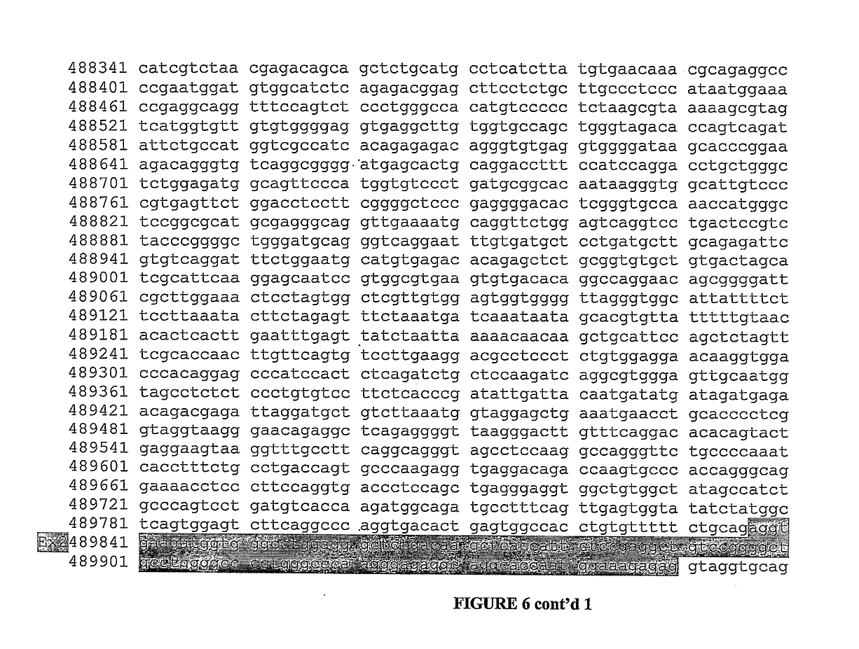

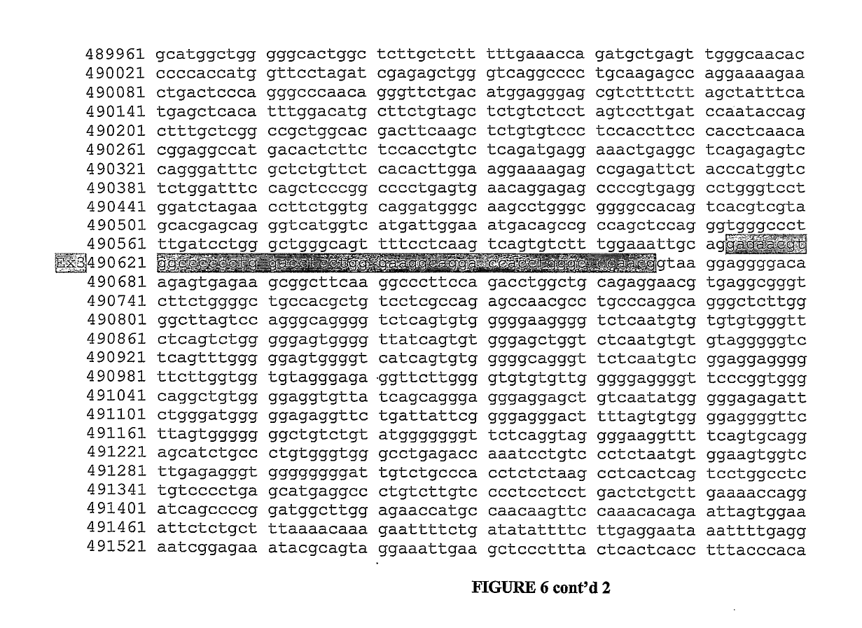

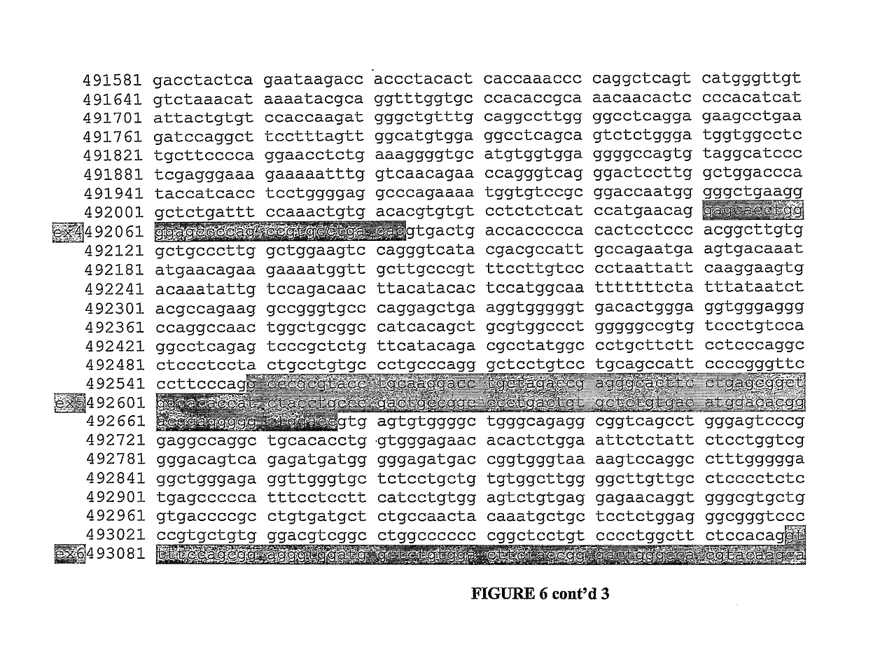

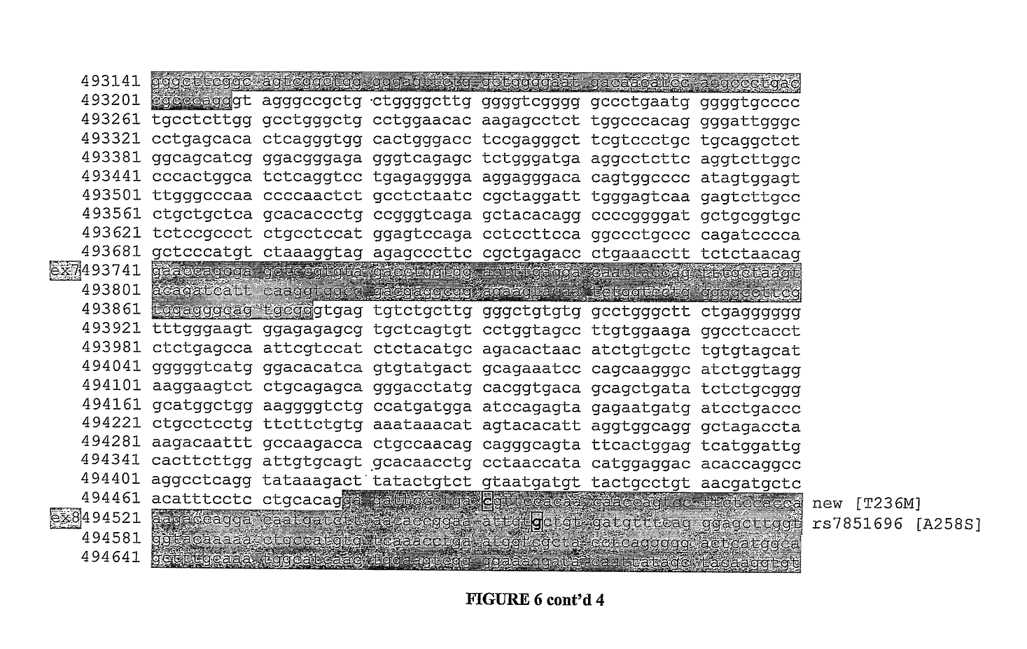

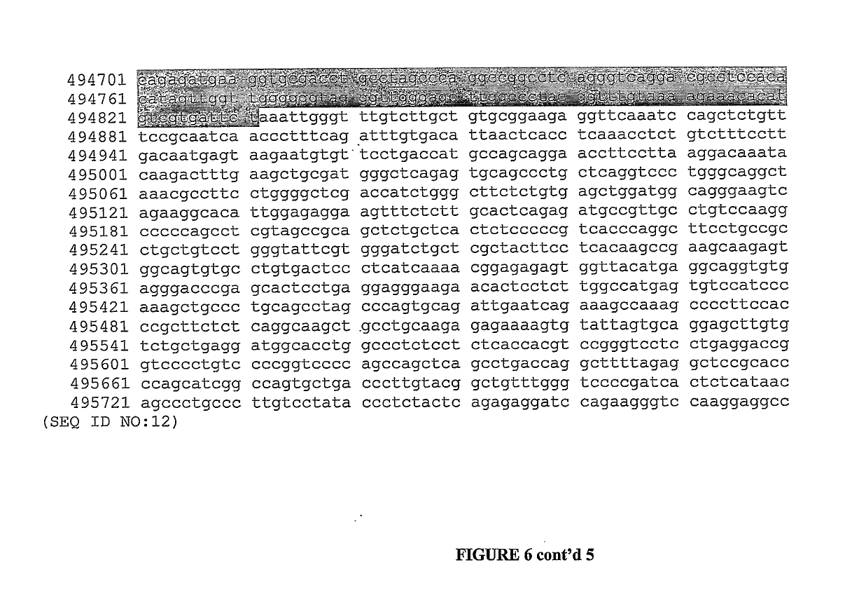

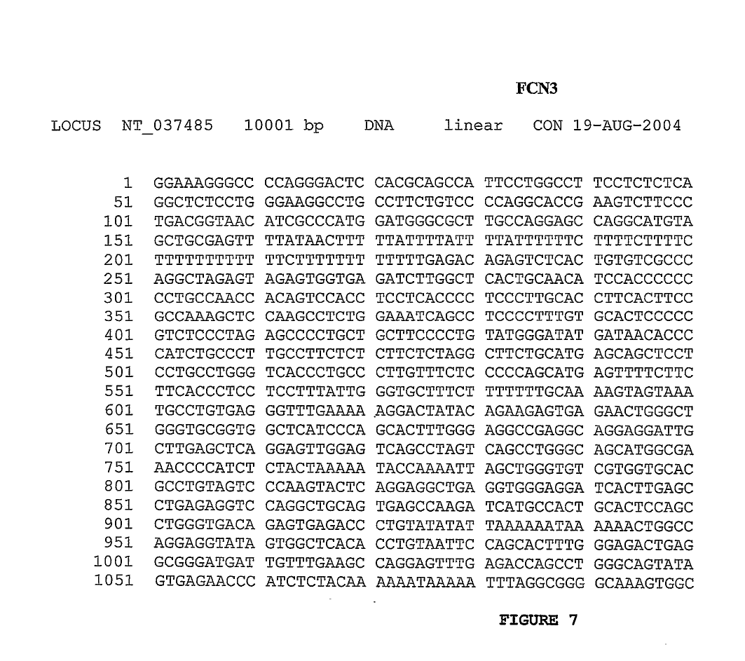

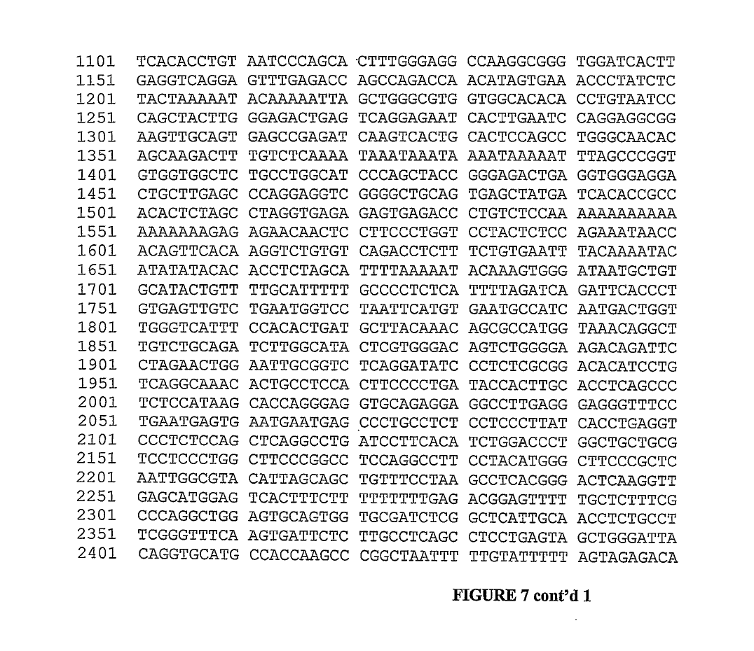

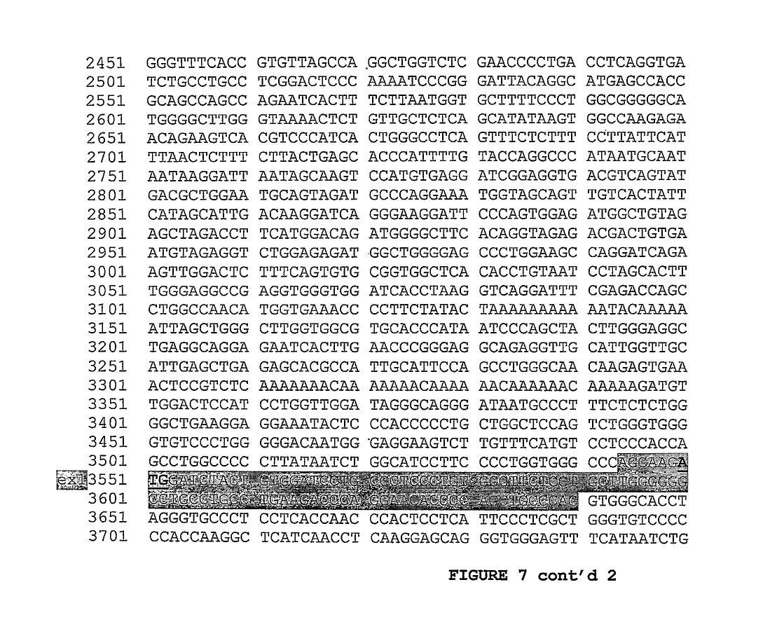

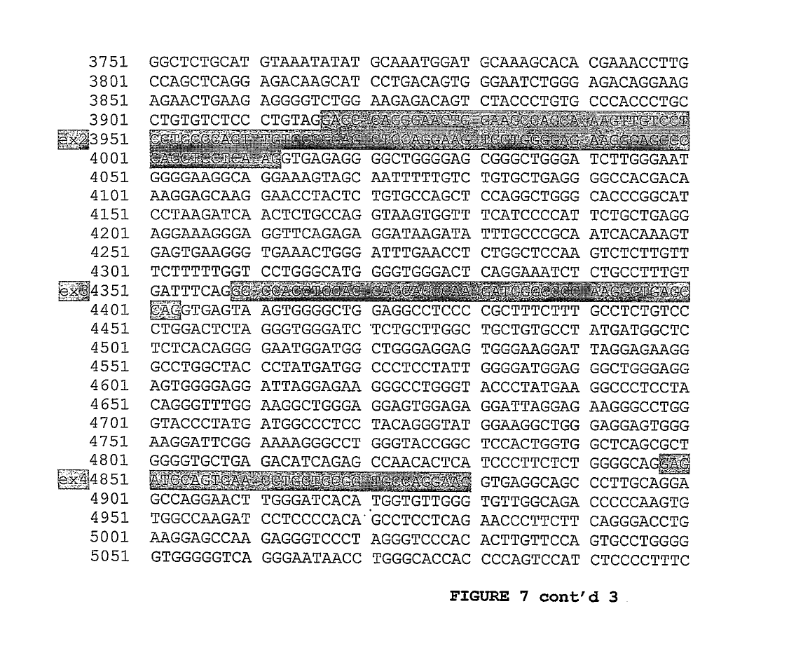

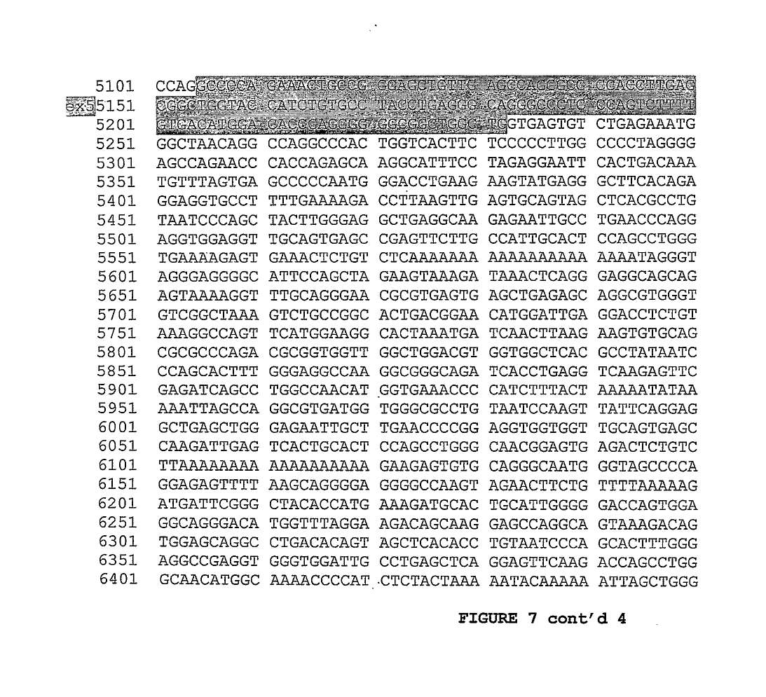

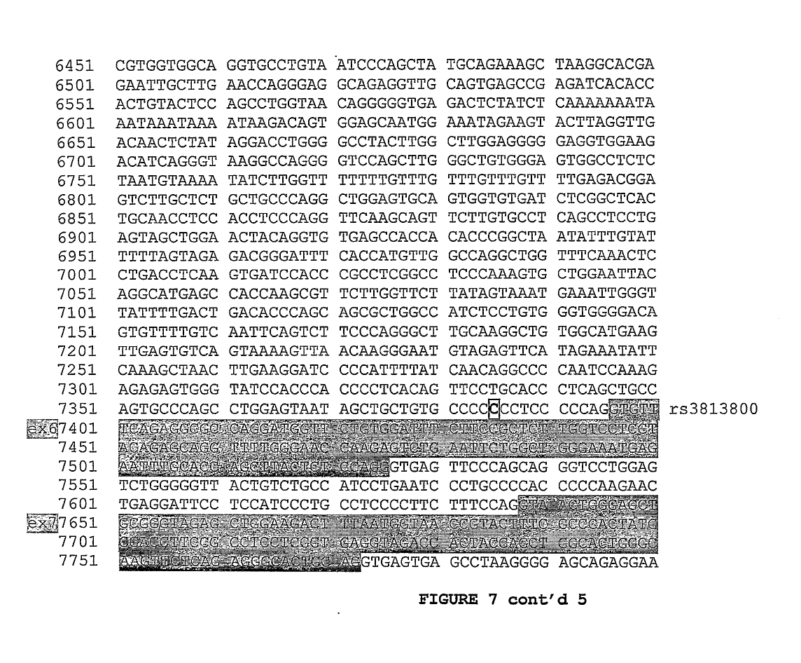

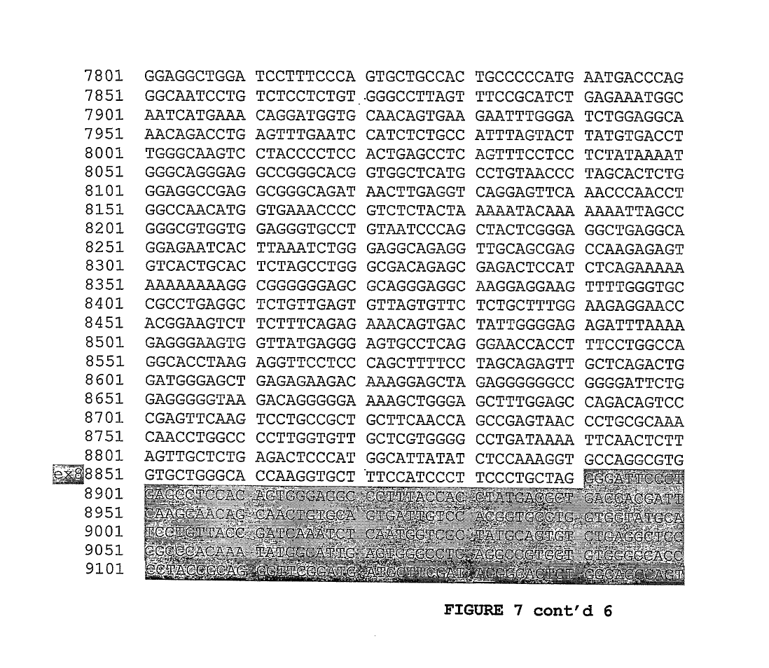

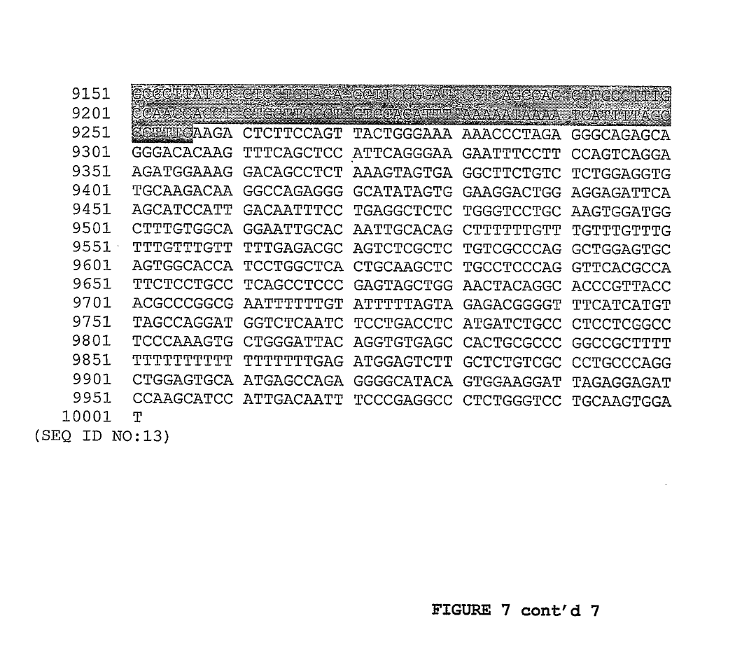

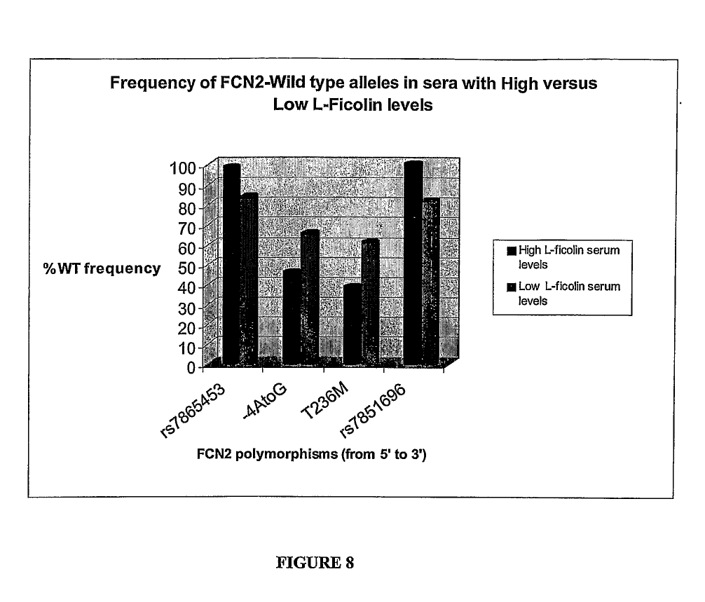

The structure of the FCN1 gene, the gene encoding the human ficolin 1 protein, is shown in FIGS. 1 and 5. The FCN1 gene has 9 exons. Exon 9 contains a nucleic acid variant at position 7919 of the gDNA sequence. More specific, the nucleic acid variant is 7919A>G and results in the amino acid Q275Q and is identified as rs1071583 (dbSNP NCBI).

The promotor region of the FCN1 gene is also polymorphic. A nucleic acid variant is present at nucleic acid position −1981 of the gDNA sequence. More specific, the nucleic acid variant is −1981C>T (complement G>A) and is identified as rs2989727. Within the FCN1 gene, a common haplotype has been found using two DNA variations included in this invention. This haplotype was found in approximately 41% of investigated individuals.

### FCN1 Haplotype: FCN1-2989727 A allele - - - FCN1-rs1071583 G allele

The structure of the FCN2 gene, the gene encoding the human ficolin 2 protein, is shown in FIGS. 2 and 6. The FCN2 gene has 8 exons. Exon 8 contains two nucleic acid variants, i.e. at nucleic acid position 6359 and 6424 of the gDNA sequence. More specific, the nucleic acid variants are:
6359C>T which results in the amino acid change T236M; and
6424G>T which results in the amino acid change A258S and is identified as rs7851696.

The promotor region of the FCN2 gene is also polymorphic. A nucleic acid variant is identified at position −64 of the gDNA sequence. More specific, the nucleic acid variant is −64A>C and is identified as rs7865453. A further nucleic acid variant is identified in the 5′UTR of the FCN2 gene, i.e. at position 4 of the gDNA sequence. More specific, the nucleic acid variant is 4A>G.

Within the FCN2 gene, two common haplotypes were found using four DNA variations included in this invention.

Haplotype FCN2.1 occurred in 23% of normal individuals and is presented by:

### FCN2-rs7865453 C allele - - - FCN2 rs7851696 T allele

A second haplotype FCN2.2 occurred in 22% of normal individuals and is presented by: FCN2-4A>G G-allele - - - FCN2 ex8-T236M T allele: in 22%

The structure of the FCN3 gene, the gene encoding the human ficolin 3 protein, is shown in FIGS. 3 and 7. The FCN3 gene has 8 exons. The IVS5 region of the human ficolin 3 gene is polymorphic. A nucleic acid variant is present at nucleic acid position 3836 of the gDNA sequence. More specific, the nucleic acid variant is 3836C>G and is identified as rs3813800.

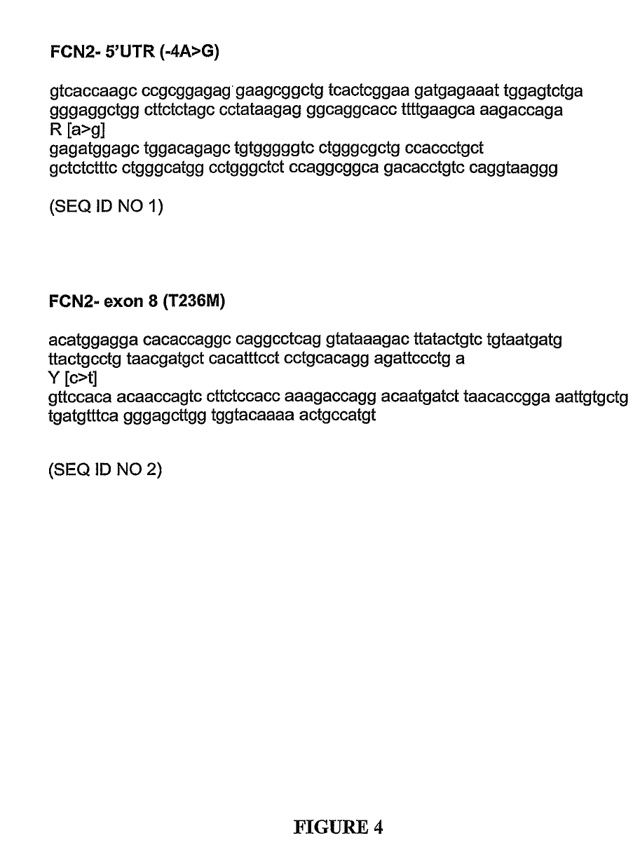

The nucleic acid variants at positions 4 and 6359 of the gDNA sequence of the FCN2 gene have been identified for the first time in the present invention. The flanking sequences of the nucleic acid variants are given in FIG. 4.

Accordingly, the present invention also relates to a method for detecting the presence or absence of a nucleic acid variant in the FCN2 gene comprising determining a nucleotide at position 4 and/or position 6359 in the FCN2 gene as defined by NT_{—}019501 (gDNA; Version: NT_{—}019501.12, GI:29731701; SEQ ID NO:12), and thereby detecting the presence or absence of the nucleic acid variant.

As described herein, any method can be used for determining the presence or absence of the nucleic acid variant. In a specific embodiment, the nucleic acid variant is determined by sequencing, hybridization, PCR, primer extension, MLPA, OLA or restriction site analysis.

More particular, the invention relates to an isolated nucleic acid comprising at least 8 nucleotides and specifically hybridizing with the wild type or variant sequence of the FCN2 gene including position 4 and/or 6359, or the complement thereof.

More preferably, the nucleic acid comprises at least 9, 10, 11, 12, 13, 14 or 15 nucleotides and up to 40, 30, 25, 24, 23, 22, 21, or 20 nucleotides. Even more preferably, the nucleic acid consists of 10 to 25, 30, 35 or 40 nucleotides. Said nucleic acids can be used as a primer or probe. In general such primers or probes will comprise nucleotide sequences entirely complementary to the corresponding wild type or variant locus in the ficolin gene.

Examples of suitable primers and probes are given in Tables 1 and 2.

TABLE 1

| | | | |
|---|---|---|---|
| Gene | Region | Forward primer | Reverse primer |

| | | | |
|---|---|---|---|
| FCN2 | 5′UTR | ctttgcccaaggccacaagc | ggaggccctggagtgccc |
| | | (SEQ ID NO 3) | t |
| | | | (SEQ ID NO 4) |
| | exon8 | acatggaggacacacggcc | gctgccatgagtccccct |
| | | (SEQ ID NO 5) | ga |
| | | | (SEQ ID NO 6) |

TABLE 2

| | | | | | |
|---|---|---|---|---|---|
| Gene | Region | Snp | Nt | Wt probe | Mutant probe |

| | | | | | |
|---|---|---|---|---|---|
| FCN2 | FCN2-promoter | 5′UTR-4 | A/G | aagaccagaagagatggagc | gctccatctcctctggtctt |
| | | | | (SEQ ID NO 7) | (SEQ ID NO 8) |
| | FCN2-Ex8 | T236M | C/T | attccctgacgttccacaac | gttgtggaacatcagggaat |
| | | | | (SEQ ID NO 9) | (SEQ ID NO 10) |

Accordingly, the present invention relates to a primer or probe comprising or consisting of the nucleic acid sequence as identified by SEQ ID Nos 3-10, or the complementary thereof.

However, if required one or more nucleotides may be added or deleted and one or more mismatches may be introduced, provided that the discriminatory power of the oligonucleotide primer or probe is not unduly affected. Accordingly, oligonucleotides consisting of 10, 11, 12, 13, 14 or 15 nucleotides and up to 25, 26, 27, 28, 29 or 30 nucleotides and having a sequence for 80, 85, 90 or 100% identical with the sequences of SEQ ID Nos 3-10 are also encompassed by the present invention. Specific length and sequence of the probes and primers will depend on the complexity of the required nucleic acid target, as well as on the reaction conditions such as temperature and ionic strength. In general, the hybridization conditions are to be stringent as known in the art. “Stringent” refers to the condition under which a nucleotide sequence can bind to related or non-specific sequences. For example, high temperature and lower salt increases stringency such than non-specific binding or binding with low melting temperature will dissolve.

The present invention furthermore relates to a method for detecting the presence or absence of a nucleic acid variant at position 4 and/or 6359 in the FCN2 gene comprising the amplification of the region comprising position 4 and/or 6359 using at least one specific pair of primers specifically hybridizing to the sequence as represented by NT_{—}019501 (SEQ ID NO:12), or the complementary thereof.

In a further embodiment, the present invention also comprises a kit for detecting the presence or absence of a nucleic acid variant at position 4 and/or 6359 in the FCN2 gene comprising:
at least one oligonucleotide probe suitable for detection of a target ficolin polynucleic acid and/or at least one oligonucleotide pair suitable for amplification of a target ficolin polynucleic acid;
when appropriate, an agent for denaturing nucleic acids;
when appropriate, an enzyme capable of modifying a double stranded or single stranded nucleic acid molecule;
when appropriate, a hybridization buffer, or components necessary for producing said buffer;
when appropriate, a wash solution, or components necessary for producing said solution;
when appropriate, a means for detecting partially or completely denatured polynucleic acids and/or a means for detecting hybrids formed in the preceding hybridization and/or a means for detecting enzymatic modifications of nucleic acids; or
when appropriate, a means for attaching an oligonucleotide to a known location on a solid support.

As used herein, the term “wild-type” sequence is analogous to the reference sequence. The nucleic acid sequence of the wild type human FCN1, FCN2 and FCN3 gene is identified by respectively SEQ ID NO:11, 12 and 13, or the complementary thereof. For example, the allele may be normal as in the reference sequence, or it may be a variant, such as a structural or a non-structural variant.

In the present context “ficolin” also covers analogues of ficolin. An analogue is a compound (or molecule) that is a (chemical) structural derivative of ficolin. It is also used to describe a molecule which may be structurally similar (but not identical) to another, and which exhibits many or some of the same biological functions of ficolin. An analogue is to be understood as being any peptide sequence capable of the same biological functions as wild-type ficolin, including recombinant ficolin.

As used herein, the term “innate immunity” refers to the natural ability of an organism to defend itself against invasions by pathogens. Pathogens as used herein, may include, but are not limited to bacteria, fungi, parasites, viruses and algae. In addition, innate immunity includes immune responses that affect other diseases, such as cancer, inflammatory diseases, autoimmune diseases, various infections, and the like.

As used herein, an “indication or condition associated with aberrant, modified or altered innate immunity” refers to any indication or disease resulting from a decreased or increased defense mechanism. A decreased defense can increase or alter the susceptibility for infection or inflammation or can increase risk for acquiring of a particular disease. An increased defense might result in autoimmune disease or inflammatory diseases.

It has been determined in the present invention that ficolin deficiencies are associated with an increased risk for infections, inflammation and autoimmune conditions, and influence the severity and/or course of several diseases. Accordingly, ficolin deficiencies can be linked with increased susceptibility for disease and/or prognosis for more severe or more frequent disease, or worse outcomes due to complication. In general, ficolin deficiencies can be linked with altered activity of innate immunity. Furthermore, treatment options can be considered and include eventual ficolin replacement therapy. Alternatively, it is shown in the present invention that ficolin deficiencies can also be associated with a reduced (i.e. lower or no) risk for infections, inflammation or autoimmune conditions, indicating the protective effect of the ficolin deficiency.

With the methods of the present invention, the risk for developing a disorder associated with an altered activity of innate immunity can be determined.

Accordingly, the present invention relates to a method of identifying a subject at risk of, or having, an indication associated with altered innate immunity, comprising detecting the presence or absence of at least one nucleic acid variant in at least one of the ficolin genes.

More specific, the present invention relates to a method of identifying a subject at risk of, or having, an indication associated with altered innate immunity, comprising detecting the presence or absence of at least one nucleic acid variant in at least one genes selected from the group consisting of:
the FCN1 gene,
the FCN2 gene, and
the FCN3 gene,
   whereby the presence of at least one nucleic acid variant identifies a subject at risk of, or having an increased or decreased susceptibility for disease. In the latter case, the nucleic acid variant has a protective effect on the risk of developing a disease.

In a further embodiment, the presence of at least one ficolin nucleic acid variant identifies a subject at risk of, or having an increased severity of disease. In an even further embodiment, the presence of at least one ficolin nucleic acid variant identifies a subject at risk of, or having a modified response to therapy for a disease.

Furthermore, the presence of at least one ficolin nucleic acid variant identifies a subject at risk of, or having increased risk of transplant rejection.

More specific, the altered innate immunity is associated with an increased susceptibility for infection, autoimmune disease, cystic fibrosis, cardiovasular disease, Alzheimer's disease or cancer.

As used herein, the term “infection” encompasses bacterial, viral, fungal, parasitic or algae infection. Sepsis is defined as presence of infection (documented or suspected) and several of other parameters of general clinical nature, inflammatory, hemodynamic and tissue perfusion parameters. Severe sepsis is the presence of sepsis complicated by organ dysfunction. Septic shock is defined as the presence of severe sepsis accompanied by acute circulatory failure. Otitis media is an infection of the middle ear.

In a specific embodiment, the altered innate immunity is associated with one or more of the following autoimmune diseases: rheumatoid arthritis (RA), spondyloarthropathy, systemic lupus erythematosus (SLE), Sjogren's disease, multiple sclerosis (MS), Crohn's disease, coeliac disease, Type 1 diabetes, Kawasaki disease, asthma, atopic dermatitis, dermatomyositis or Behçet's disease.

In a further embodiment, the altered innate immunity is associated with one or more of the following cancers: (1) solid tumors such as colon cancer, colorectal cancer, gastric cancer, cervical cancer, lung cancer, liver cancer, kidney cancer or brain cancer, and (2) haematological malignancies such as a) Leukemias: acute myeloid leukemia (AML), acute lymphoid leukemia (ALL), chronic myeloid leukemia (CML) and chronic lymphoid leukemia (CLL), b) Lymphomas: Hodgkins and non-Hodgkin's lymphomas and c) Myelomas.

In a more specific embodiment, the altered innate immunity is associated with one or more of the following cardiovascular diseases: bypass failure, atherosclerosis, myocardial reperfusion injury, coronary artery disease or heart disease.

In a particular embodiment, the altered innate immunity is associated with an increased severity of disease influencing the course of a disease. For example, in the case of infection the disease status can be aggravated leading to a higher mortality. In autoimmune disease, for example RA, the severity or damage to the joints can be more pronounced, as measured by radiology.

In a further embodiment, the altered innate immunity is associated with a modified response to therapy for a specific disease resulting in adverse effects. This can for example by seen in vaccinations or in NSAID therapy.

In a particular embodiment, the present invention relates to a method of identifying a subject at risk of, or having an increased susceptibility for (recurrent) otitis media, rheumatoid arthritis, recurrent infections, sepsis, severe sepsis or septic shock, comprising detecting the presence or absence of at least one nucleic acid variant in at least one of the FCN genes.

Furthermore, the present invention also relates to a method for determining a protective effect on the risk of developing (recurrent) otitis media, rheumatoid arthritis, recurrent infections, sepsis, severe sepsis or septic shock, comprising detecting the presence or absence of at least one nucleic acid variant in at least one of the FCN genes.

As used herein, “deficiency” refers to an alteration in the genomic DNA sequence when compared to the wild type sequence. This may result in altered (enhanced or decreased) expression levels or may result in changes (increase or decrease) in functionality of the encoded protein, or even may result in a change of function of the protein (“gain of function”).

The “subject” on which the method of the present invention is carried out can be any subject of which the risk of an altered innate immunity needs to be determined. The subject may be a non-human subject such as (but not limited to) a cow, a pig, a sheep, a goat, a horse, a monkey, a rabbit, a dog, a cat, a mouse, a rat, a hamster, a zebrafish, a pufferfish (Fugu), a fly, a worm or C. elegans. More preferably, the subject is a primate. Even more preferably, the subject is a human.

It will be apparent to the person skilled in the art that there are a large number of analytical procedures which may be used to detect the presence or absence of the nucleic acid variants mentioned herein. Nucleic acid from any nucleated cell can be used as the starting point for such assay techniques and may be isolated according to standard nucleic acid preparation procedures well known to those of skill in the art. Many current methods for the detection of allelic variation are reviewed by Nollau et al. (1997), and in standard textbooks, for example “Laboratory Protocols for Mutation Detection”, Ed. by U. Landegren, Oxford University Press, 1996 and “PCR”, 2^{nd} Edition” by Newton & Graham, BIOS Scientific Publishers Limited, 1997 (incorporated herein by reference).

The method of the present invention can be carried out in vivo or in vitro. Preferred, however, is in vitro detection of nucleic acid variants in the ficolin gene in a biological sample obtained from the subject. The term “biological sample” means a tissue sample or a body fluid sample. A tissue sample includes (but is not limited to) buccal cells, a brain sample, a skin sample or organ sample (e.g. liver). The term “body fluid” refers to all fluids that are present in the body including but not limited to blood, plasma, serum, lymph, urine, saliva or cerebrospinal fluid. The biological sample may also be obtained by subjecting it to a pretreatment if necessary, for example, by homogenizing or extracting. Such a pretreatment may be selected appropriately by those skilled in the art depending on the biological sample to be subjected.

A nucleic acid comprising an intended sequence prepared from a biological sample may be prepared from DNA (e.g. gDNA or cDNA) or RNA (e.g. mRNA). Release, concentration and isolation of the nucleic acids from the sample can be done by any method known in the art. Currently, various commercial kits are available such as the QIAamp Blood Kit from Qiagen (Hilden, Germany) for the isolation of nucleic acids from blood samples, or the ‘High pure PCR Template Preparation Kit’ (Roche Diagnostics, Basel, Switzerland) or the DNA purification kits (PureGene, Gentra, Minneapolis, US). Other, well-known procedures for the isolation of DNA or RNA from a biological sample are also available (Sambrook et al., 1989; Ausubel et al., 2003).

When the quantity of the nucleic acid is low or insufficient for the assessment, the nucleic acid may be amplified. Such amplification procedures can be accomplished by those methods known in the art, including, for example, the polymerase chain reaction (PCR), ligase chain reaction (LCR), nucleic acid sequence-based amplification (NASBA), strand displacement amplification, rolling circle amplification, T7-polymerase amplification, and reverse transcription polymerase reaction (RT-PCR).

After performing the extraction and/or amplification procedure, the presence or absence of certain nucleic acid variants in the target sequence can be detected. Numerous methods for detecting a single nucleotide anomaly in nucleic acid sequences are well-known in the art. The present invention is not limited by any particular method used to detect the target sequences disclosed herein. Examples of such methods are described by Gut (2001) and Syvänen (2001), and include, but are not limited to, hybridization methods such as reverse dot blot, LiPA, genechip microarrays, DASH, PNA and LNA probes, TaqMan (5′nuclease assay) and molecular beacons; allele-specific PCR methods such as intercalating dye, FRET primers and Alphascreen; primer extension methods such as ARMS, kinetic PCR, SNPstream, GBA, multiplex minisequencing, SNaPshot, pyrosequencing, MassExtend, MassArray, Goodassay, microarray miniseq, APEX, sequence specific priming (SSP), microarray primer extension, Tag arrays, coded microspheres, TDI, fluorescence polarization; oligonucleotide ligation methods such as colorimetric OLA, sequence-coded OLA, microarray ligation, ligase chain reaction, padlock probes and rolling circle amplification; endonuclease cleavage methods such as restriction site analysis (RFLP) and Invader assay.

In a preferred embodiment, the detection of the presence or absence of a nucleic acid variant is determined by hybridization, sequencing, PCR, primer extension, MLPA, OLA or restriction site analysis.

The present invention also provides oligonucleotides, i.e. primers and probes, in order to amplify and/or detect nucleic acid variants and/or the wild type sequence of the ficolin genes, i.e. FCN1, FCN2 and/or FCN3. The wild type sequence of the FCN genes are identified by SEQ ID NO:11, 12 and 13. Such primers or probes, specifically hybridizing to the target nucleic acid, are of any convenient length such as to comprise at least 8, 9, 10, 11, 12, 13, 14 or 15 nucleotides and up to 40 nucleotides, up to 30 nucleotides or more conveniently up to 25 nucleotides in length, such as for example 8, 9, 10, 11, 12, 13, 14, 15, 16, 17, 18, 19, 20, 21, 22, 23, 24 or 25 nucleotides in length. In general such primers or probes will comprise nucleotide sequences entirely complementary to the corresponding wild type or variant locus in the ficolin gene. However, if required one or more nucleotides may be added or one or more mismatches may be introduced, provided that the discriminatory power of the oligonucleotide primer or probe is not unduly affected. Specific length and sequence of the probes and primers will depend on the complexity of the required nucleic acid target, as well as on the reaction conditions such as temperature and ionic strength. In general, the hybridization conditions are to be stringent as known in the art. The primers or probes of the invention may carry one or more labels to facilitate detection.

In a preferred embodiment, the primer or probe consists of 10 to 30 nucleotides, preferably 15 to 30 nucleotides, and is capable of specifically forming a hybrid with a part of the FCN gene and is at least one or more selected from the group consisting of:
1) an oligonucleotide capable of hybridizing under a stringent condition with the sequence as represented by SEQ ID NO:11, 12 or 13, or the complementary thereof;
2) an oligonucleotide of which the sequence is for 80, 85 or 90% identical to the sequence as represented by SEQ ID NO:11, 12 or 13, or the complementary thereof; and
3) an oligonucleotide capable of hybridizing under a stringent condition with the sequence as represented by SEQ ID NO:11, 12 or 13 wherein one or more nucleotides was subjected to a variation such as a substitution, deletion, insertion or addition, or the complementary thereof.

More particular, the present invention relates to an isolated oligonucleotide consisting of 10 to 30 nucleotides, optionally 15 to 30 nucleotides, for detecting the presence of one or more nucleic acid variants in SEQ ID NO:11, 12 or 13, or the complementary strand. More specific, the nucleic acid variants are located at position −1981 and 7919 of SEQ ID NO:11, position −64, −4, 6424 and 6359 of SEQ ID NO:12 and position 3836 of SEQ ID NO:13.

The polymorphism located in the FCN gene may also be detected in vitro by determining in the isolated FCN protein, as identified in the present invention, the presence or absence of an amino acid change by sequencing said protein. The amino acid change may also be detected by any conventional method known in the art, for example by mass-spectroscopy, gel electrophoresis, MALDI-TOF mass spectroscopy, ELISA, protein arrays, determination of the molecular weight, or by isoelectrofocusing.

Studies have demonstrated several other known risk factors for altered innate immunity. Any human gene can be studied together with the method of the present invention. Of the different genetic markers identified, further important risk factors are polymorphisms or nucleic acid variations in one or more of the following genes: BPI (Bacterial/permeability-increasing protein), CD14 (CD14 antigen precursor), beta-catenin (CTNNBI, Cadherin Associated Protein beta I), IL10 (Interleukin 10), LBP (Lipopolysaccharide binding protein), RP105 (LY64, lymphocyte antigen 64 homolog radioprotective), MBL2 (Mannose Binding Protein), MD-1 (RP105-associated), MD-2 (MD2 Protein, Lymphocyte antigen 96), MYD88 (Myeloid differentiation primary response gene 88), NOD1 (Caspase recruitment domain 4, CARD4), NOD2 (Caspase recruitment domain family, member 15, CARD15) and the Toll Like Receptor genes, i.e. TLR1, TLR2, TLR3, TLR4, TLR5, TLR6, TLR7, TLR8, TLR9 and TLR10.

Accordingly, the present invention also relates to a method of identifying a subject at risk of, or having, an indication associated with altered innate immunity, comprising the step of detecting the presence or absence of a nucleic acid variant in at least one of the ficolin genes and detecting the presence or absence of one or more nucleic acid variants in any other gene, and more specific in the genes selected from the group consisting of: BPI (Bacterial/permeability-increasing protein), CD14 (CD14 antigen precursor), beta-catenin (CTNNBI, Cadherin Associated Protein beta), IL10 (Interleukin 10), LBP (Lipopolysaccharide binding protein), RP105 (LY64, lymphocyte antigen 64 homolog radioprotective), MBL2 (Mannose Binding Protein), MD-1 (RP105-associated), MD-2 (MD2 Protein, Lymphocyte antigen 96), MYD88 (Myeloid differentiation primary response gene 88), NOD1 (Caspase recruitment domain 4, CARD4), NOD2 (Caspase recruitment domain family, member 15, CARD15), and the Toll Like Receptor genes, i.e. TLR1, TLR2, TLR3, TLR4, TLR5, TLR6, TLR7, TLR8, TLR9 or TLR10, possibly in combination with the detection of one or more other risk factors.

The presence of nucleic acid variants in the genes of a subject may also be reflected in the concentration, structure and functionality of the ficolin protein in the serum or plasma of said subject. Therefore, the present invention also encompasses a method for determining whether a subject has a risk of developing a disease wherein the nucleic acid variants in the ficolin genes are detected by their protein phenotype. The invention discloses how decreased or increased levels of ficolin and lack of functional ficolin is crucial in the innate immunity defense. In a specific embodiment, the method encompasses the measurement of one or more proteins.

Accordingly, the present invention relates to a method for identifying a subject at risk of, or having, an indication associated with altered innate immunity comprising the steps of:
a) determining the concentration or functionality of a ficolin protein in a sample,
b) identifying if said subject is at risk of, or has, an indication associated with altered innate immunity.

More particular, the current invention provides a method of identifying a subject at risk of, or having, an indication associated with altered innate immunity, comprising measuring the concentration or functionality of at least one ficolin protein in a biological sample, wherein an increased or decreased ficolin concentration or altered functionality compared to a reference value representing a known health status indicates that said subject is at risk of or suffers from a disorder associated with altered innate immunity.

As used herein, a “ficolin protein” is a protein encoded by one of the FCN1, FCN2 or FCN3 genes as described in the present invention, or variants thereof.

The term “concentration” or “level”, as used in the present invention, refers to the presence or absence and/or amount of a certain protein. A change in the concentration of a protein refers to a measurable increase or decrease, including total absence or presence, in the protein concentration when compared to a control subject. “A known health status” or “control subject”, as defined in the present invention is a subject of the same species as the subject under examination which is free from, or not at direct risk of developing a disease, more specific an infection or inflammation. The healthy subject can be of the same weight, age, and gender as the subject who is being diagnosed or prognosed for an altered innate immunity. In some cases, it might be preferred to use a reference value from the subject which is diagnosed. For any given ficolin protein, the concentration obtained upon analyzing the subject under examination relative to the concentration obtained upon analyzing a control subject will depend on the particular analytical protocol and detection technique that is used. Accordingly, those skilled in the art will understand that, based on the present description, any laboratory can establish, for a given ficolin protein, a suitable “reference range”, “reference level range”, “concentration range or “range of levels” (those terms are used interchangeable) characteristic for control subjects according to the analytical protocol and detection technique in use. The concentration obtained for the subject under examination can then be compared with this reference range and based on this comparison, a conclusion can be drawn as to whether the subject has a risk of developing a disease as described herein. Those skilled in the art will also know how to establish, for a given ficolin protein, a cut-off value suitable for determining whether a subject is at risk of, or has, an altered innate immunity. Methods for defining cut-off values include (but are not limited to) the methods described by IFCC (1987). In a preferred embodiment, the reference value can be that of a level or concentration of a ficolin protein in a sample, preferably a body fluid, from a subject not suffering from a disease, more specific an infection or inflammation. The healthy subject can be of the same weight, age, and gender as the subject who is being diagnosed or prognosed for an altered innate immunity. In some cases, it might be preferred to use a reference value from the subject which is diagnosed.

The ficolin proteins that are detected in the method of the present invention, may be detected by any method known to those skilled in the art. They can be identified by their structure, by partial amino acid sequence determination, by functional assay, by enzyme assay, by various immunological methods, or by biochemical methods known to those skilled in the art.

The functional assay may encompass the measurement of the ability to opsonize heat-killed baker's yeast (Miller et al., 1968), the assessment of phagocytosis of various microorganisms (Kuhlman et al., 1989) and/or detection of complement activation (Super et al., 1989; 1990; Yokota et al., 1995). In a serum test for complement activation described by Seelen et al. (2003), for example, the lectin pathway function is assessed using plates coated with mannan, followed by incubation of the serum in buffer containing Ca²⁺, Mg²⁺ and an inhibitory antibody directed against C1q. The formation of the membrane attack complex is subsequently detected by use of a specific monoclonal antibody directed against C5b-9.

Biochemical methods include (but are not limited to) capillary electrophoresis, high performance liquid chromatography (HPLC), thin layer chromatography (TLC), hyperdiffusion chromatography, two-dimensional liquid phase electrophoresis (2-D-LPE; Davidsson et al. 1999) or detection of the migration pattern in gel electrophoreses. Sodium dodecyl sulfate-polyacrylamide gel electrophoresis (SDS-PAGE) is a widely used approach for separating proteins from complex mixtures (Patterson and Aebersold, 1995). It can be performed in one- or two-dimensional (2-D) configuration. For less complicated protein preparation, one-dimensional SDS-PAGE is preferred over 2-D gels, because it is simpler. However, SDS-PAGE often results in migrating or overlapping protein bands due to its limited resolving power. What appears to be a single band may actually be a mixture of different proteins. 2-D gel electrophoresis incorporates isoelectric focusing (IEF) in the first dimension and SDS-PAGE in the second dimension, leading to a separation by charge and size (O'Farrell, 1975). 2-D PAGE is a powerful technique for separating very complex protein preparations, resolving up to 10 000 proteins from mammalian tissues and other complex proteins (Klose and Kobalz, 1995; Celis et al., 1996; Yan et al., 1997). The ficolin proteins of the present invention can be identified by their isoelectric focusing point (pI) and their molecular weight (MW) in kilodaltons (kD).

As indicated above, the level of ficolin protein can also be detected by an immunoassay. As used herein, an “immunoassay” is an assay that utilizes an antibody to specifically bind to the antigen (i.e. the ficolin protein). The immunoassay is thus characterized by detection of specific binding of a ficolin protein to an antibody. Immunoassays for detecting ficolin proteins may be either competitive or noncompetitive. Noncompetitive immunoassays are assays in which the amount of captured analyte (i.e. the ficolin protein) is directly measured. In competitive assays, the amount of analyte (i.e. the ficolin protein) present in the sample is measured indirectly by measuring the amount of an added (exogenous) analyte displaced (or competed away) from a capture agent (i.e. the antibody) by the analyte (i.e. the ficolin protein) present in the sample. In one competition assay, a known amount of the (exogenous) ficolin protein is added to the sample and the sample is then contacted with the antibody. The amount of added (exogenous) ficolin protein bound to the antibody is inversely proportional to the concentration of the ficolin protein in the sample before the exogenous ficolin protein is added. In one preferred “sandwich” assay, for example, the antibodies can be bound directly to a solid substrate where they are immobilized. These immobilized antibodies then capture the ficolin protein of interest present in the test sample. Other immunological methods include but are not limited to fluid or gel precipitation reactions, immunodiffusion (single or double), agglutination assays, immunoelectrophoresis, radioimmunoassays (RIA), enzyme-linked immunosorbent assays (ELISA), TRIFMA (Christiansen et al., 1999), Western blots, liposome immunoassays (Monroe et al., 1986), complement-fixation assays, immunoradiometric assays, fluorescent immunoassays, protein A immunoassays or immunoPCR. An overview of different immunoassays is given in Wild (2001), Ghindilis et al. (2002) and Kilpatrick (2002).

In a further embodiment, the method of the present invention may also be used in determining whether and which therapeutic agent might be suitable for a patient being at risk of, or having an indication associated with altered innate immunity. The therapeutic agent may be used to prevent or treat the indication or disease. As used herein, the term “preventing a disease” means inhibiting or reversing the onset of the disease, inhibiting or reversing the initial signs of the disease, inhibiting the appearance of clinical symptoms of the disease. As used herein, the term “treating a disease” includes substantially inhibiting the disease, substantially slowing or reversing the progression of the disease, substantially ameliorating clinical symptoms of the disease or substantially preventing the appearance of clinical symptoms of the disease.

Another aspect of the invention relates to a kit for identifying a subject at risk of, or having, an indication associated with altered innate immunity. This kit can be based on the detection of nucleic acid variants in the ficolin gene of said subject or it can be based on the detection of ficolin proteins. Accordingly, the kit of the present invention comprises reagents that selectively detect a nucleic acid variant in the ficolin gene or that selectively detect a ficolin protein.

A kit based on the detection of nucleic acid variants in the ficolin gene may comprise:
(a) a means for detecting the presence or absence of one or more nucleic acid variants in the ficolin genes of said subject; and/or
(b) a means for determining, from the nucleic acid variants detected with the means of step (a), whether the subject is at risk of, or has, an indication associated with altered innate immunity.

More preferred, the kit comprises a means for detecting the presence or absence of one or more nucleic acid variants in:
the promotor region and/or exon 9 of the FCN1 gene,
the promotor region, the 5′UTR and/or exon 8 of the FCN2 gene, or
the IVS5 region of the FCN3 gene.

In a preferred embodiment of the present invention, the kit comprises:
(a) a mean for detecting the presence or absence of one or more nucleic acid variants at the following positions of the ficolin genes of said subject:
   position −1981 and/or at position 7919 of the genomic DNA sequence of the FCN1 gene,
   position 4, −64, 6424 and/or 6359 of the of the genomic DNA sequence of the FCN2 gene, and/or
   position 3836 of the of the genomic DNA sequence of the FCN3 gene; and
(b) a means for determining, from the nucleic acid variants detected with the means of step (a), whether the subject is at risk of, or has, an indication associated with altered innate immunity.

In a specific embodiment the means or reagents in step (a) of said kit may comprise:
(i) when appropriate, a means for obtaining a target ficolin polynucleic acid present in a biological sample and/or obtaining the nucleotide sequence thereof;
(ii) at least one oligonucleotide suitable for detection of a target ficolin polynucleic acid and/or at least one oligonucleotide pair suitable for amplification of a target ficolin polynucleic acid;
(iii) when appropriate, an agent for denaturing nucleic acids;
(iv) when appropriate, an enzyme capable of modifying a double stranded or single stranded nucleic acid molecule;
(v) when appropriate, a hybridization buffer, or components necessary for producing said buffer;
(vi) when appropriate, a wash solution, or components necessary for producing said solution;
(vii) when appropriate, a means for detecting partially or completely denatured polynucleic acids and/or a means for detecting hybrids formed in the preceding hybridization and/or a means for detecting enzymatic modifications of nucleic acids; and
(viii) when appropriate, a means for attaching an oligonucleotide to a known location on a solid support.

In a preferred embodiment the means or reagents in step (a) of said kit comprise at least one oligonucleotide probe suitable for detection of a target ficolin polynucleic acid and at least one pair of primers suitable for amplification of a target ficolin polynucleic acid. In a specific embodiment, the target ficolin nucleic acid is located in the promotor region and/or exon 9 of the FCN1 gene, in the promotor region, the 5′UTR and/or exon 8 of the FCN2 gene or in the IVS5 region of the FCN3 gene. Even more specific, the target ficolin polynucleic acid is located at position −1981 and/or 7919 of SEQ ID NO:11, position −64, −4, 6424 and/or 6359 of SEQ ID NO:12 and position 3836 of SEQ ID NO:13.

In a specific embodiment, the oligonucleotide suitable for detection of a target ficolin polynucleic acid is a probe configured to hybridize the said ficolin polynucleic acid to form an invase cleavage structure. The cleavage structure can be detected by a cleavage agent or enzyme such as a structure-specific nuclease, a 5′nuclease, a FEN-1 endonuclease or a polymerase.

The term “hybridization buffer” means a buffer allowing a hybridization reaction between the probes and the polynucleic acids present in the sample, or the amplified products, under the appropriate stringency conditions.

The term “wash solution” means a solution enabling washing of the hybrids formed under the appropriate stringency conditions.

In a specific embodiment of the kit, the means for detecting the presence or absence of nucleic acid variants in the ficolin gene is an INVADER assay (see e.g. WO97/27214, incorporated herein by reference)

In a more specific embodiment of the kit, the means for detecting the presence or absence of nucleic acid variants in the ficolin gene is a line probe assay (LiPA; Stuyver et al., 1996; Stuyver et al., 1997; Van Geyt et al., 1998). In this embodiment, the selected set of probes is immobilized to a membrane strip in a line fashion. An alternative is the immobilization of the probes in a “dotted fashion” (dot spots; DoPA). Said probes may be immobilized individually or as mixtures to the delineated locations. The amplified ficolin polynucleic acids can be labelled with biotine, and the hybrid can then, via a biotine-streptavidine coupling, be detected with a non-radioactive colour developing system. Particularly advantageous are other systems in which different nucleic acid variants can be detected simultaneously. In this multiparameter approach, oligonucleotides may be coupled to microspheres or chips. An example of an assay that provides for simultaneous detection includes (but is not limited to) the xMAP™ technology (Luminex®, Austin, Tex., USA) and the PamGene technology (PamGene, 's-Hertogenbosch, The Netherlands).

The means in step (b) of said kit, for determining, from the nucleic acid variants in the ficolin gene detected with the means of step (a), whether the subject is at risk of, or has, an indication associated with altered innate immunity include a table, a chart, or similar, generally referred to as “a predisposition risk algorithm”, indicating the ficolin nucleic acid variants or haplotypes that confer a risk for or the existence of an indication associated with altered innate immunity. As used herein, the term “chart” refers to graphical presentation, visual aid, diagram, plan, graph, map or the like including the relevant information. The determination of the risk can be performed manually or with the use of a computer.

The kit of the present invention may include, in additions to the means of steps (a), a means for detection other risk factors, e.g. nucleic acid variants in a gene, for an indication associated with an altered innate immunity. In a preferred embodiment, the kit additionally includes a means for detecting the genotype of or a nucleic acid variant in at least one of the genes selected from the group consisting of: BPI (Bacterial/permeability-increasing protein), CD14 (CD14 antigen precursor), beta-catenin (CTNNBI, Cadherin Associated Protein beta), IL10 (Interleukin 10), LBP (Lipopolysaccharide binding protein), RP105 (LY64, lymphocyte antigen 64 homolog radioprotective), MBL2 (Mannose Binding Protein), MD-1 (RP105-associated), MD-2 (MD2 Protein, Lymphocyte antigen 96), MYD88 (Myeloid differentiation primary response gene 88), NOD1 (Caspase recruitment domain 4, CARD4), NOD2 (Caspase recruitment domain family, member 15, CARD15) and the Toll Like Receptor genes, i.e. TLR1, TLR2, TLR3, TLR4, TLR5, TLR6, TLR7, TLR8, TLR9 and TLR10.

A kit based on the detection of ficolin proteins may comprise an antibody that specifically recognizes the ficolin protein that is detected. A preferred kit for carrying out the method of the invention comprises:
an antibody (primary antibody) which forms an immunological complex with the ficolin protein to be detected;
a monoclonal antibody (secondary antibody) which specifically recognizes the ficolin protein to be detected;
a marker either for specific tagging or coupling with said secondary antibody;
appropriate buffer solutions for carrying out the immunological reaction between the primary antibody and the ficolin protein, between the secondary antibody and the primary antibody-ficolin protein complex and/or between the bound secondary antibody and the marker; or
possibly, for standardization purposes, a purified ficolin protein.

### EXAMPLES

Table 3 identifies the ficolin nucleic acid and protein variants studied in the present examples.

TABLE 3

| | | | Position | |
|---|---|---|---|---|
| Gene | Region | SNP | gDNA | protein |
| FCN1 | promoter | rs2989727 | −1981G > A | na |
| | FCN1-ex9 | rs1071583 | 7919A > G | Q275Q |
| FCN2 | promoter | rs7865453 | −64A > C | na |
| | 5′UTR | new | −4A > G | na |
| | Exon 8 | rs7851696 | 6424G > T | A258S |
| | Exon 8 | new | 6359C > T | T236M |
| FCN3 | FCN3-IVS5 | rs3813800 | 3836C > G | na |

na: not applicable

For the present examples, the statistical analysis of the data is based on the determination of odds ratios (OR) using standard procedures. An odds ratio is calculated by dividing the odds in the treated or exposed (case) group by the odds in the control group. The odds of an event are calculated as the number of events divided by the number of non-events. If the odds of an event are greater than one the event is more likely to happen than not (the odds of an event that is certain to happen are infinite); if the odds are less than one the chances are that the event won't happen (the odds of an impossible event are zero). In the present examples, the strength of association was reported as odds ratios (OR) (with 95% lower (LCL) and upper (UCL) confidence limit), indicating the factor by which the risk of developing a disorder or disease is increased (OR>1), or indicating the factor for a protective effect on the risk of developing a disorder or disease (OR<1).

The 95% confidence interval (95% CI) is the range of numerical values in which we can be confident (to a computed probability, such as 90 or 95%) that the population value being estimated will be found. Confidence intervals indicate the strength of evidence; where confidence intervals are wide, they indicate less precise estimates of effect. The larger the trial's sample size, the larger the number of outcome events and the greater becomes the confidence that the true relative risk reduction is close to the value stated. Thus the confidence intervals narrow and “precision” is increased. In a “positive finding” study the lower boundary of the confidence interval, or lower confidence limit, should still remain important or clinically significant if the results are to be accepted. In a “negative finding” study, the upper boundary of the confidence interval should not be clinically significant if you are to confidently accept this result.

EXAMPLE 1
Detection of nucleic acid variants in Ficolin genes from patients with
Recurrent Otitis Media (OM) and from control subjects

### Patients Samples

A study was carried out based on blood samples from 17 patients with recurrent Otitis Media. The control group (C) consisted of 172 healthy individuals. From each blood sample, informed consent to participate in the study is available

### Detection of Nucleic Acid Polymorphisms

To determine the presence or absence of nucleic acid variants in the FCN1 and FCN2 gene, the relevant 5′UTR, promotor and coding sequences (see Table 3) of the genes under study were amplified using biotinylated oligonucleotides. The polymorphisms were detected by use of a reverse hybridization method (Dot Probe Assay) with probes designed to recognize the polymorphisms as given in Table 3. After stringent washing at 56° C., hybridized probes were incubated with a streptavidine-alkaline phosphatase conjugate. The presence of a hybridized probe was revealed using NBIT/BCIP color development. Details on the reverse hybridization are described in Stuyver et al. (1996), Stuyver et al. (1997) and Van Geyt et al. (1998).

### Statistical Analysis

17 patients with recurrent OM and 172 healthy individuals were genotyped for 2 SNPs in FCN1 gene and 4 SNPs in the FCN2 gene.

The strength of association was reported as odds ratios (OR), indicating the factor by which the risk of developing sepsis is increased or decreased.

The 95% confidence interval (95% CI) is the interval computed from the sample data which, were the study repeated multiple times, would contain the true effect 95% of the time.

### Frequencies and Odds Ratios of FCN Genotypes in OM Patients and Control Individuals

The frequencies and odds ratios of the investigated SNPs in the relevant FCN genes in Otitis Media patients and in healthy control individuals are indicated in Table 4.

It was found that
1. the presence of the minor allele for the FCN2-4A>G polymorphism had a major protective effect on the risk of developing otitis media [OR 0.39 (95% CI 0.13-1.16)],
2. the presence of the minor allele for the FCN2-T236M polymorphism had a protective effect on the risk of developing otitis media [OR 0.78 (95% CI 0.32-1.89)]

TABLE 4
Allele frequencies and Odds Ratio's for SNPs in FCN1
and FCN 2 gene - SNPs in Otitis Media patients

| | | FCN2 | FCN2 (Ex8) |
|---|---|---|---|
| | | (Prom) −4AtoG | T236M |
| | | WT: A-allele | WT: C-allele |
| SNP | n | Mut: G-allele | Mut: T-allele |
| Controls | 172 | Total WT: 152 | Total WT: 153 |
| | | Total Mut: 52 | Total Mut: 51 |
| Otitis Media | 17 | Total WT: 30 | Total WT: 27 |
| | | Total Mut: 4 | Total Mut: 7 |

| | | |
|---|---|---|
| OR (95% CI) | 0.39 (013-1.16) | 0.78 (0.32-1.89) |

EXAMPLE 2
Detection of nucleic acid variants in the FCN genes from children with a
haematological malignancy or solid tumor and from control subjects

### Patients Samples

A study was carried out based on DNA isolated from blood samples from 277 children in an oncology clinic. The control group (C) consisted of 205 healthy individuals. From each patient, informed consent to participate in the study is available

### Detection of Nucleic Acid Polymorphisms

To determine the presence or absence of nucleic acid variants in the FCN genes, the relevant promotor and coding sequences (see Table 3) were amplified using biotinylated oligonucleotides. The polymorphisms were detected by use of a reverse hybridization method (Dot Probe Assay) with probes designed to recognize the polymorphisms as given in Table 3. After stringent washing at 56° C., hybridized probes were incubated with a streptavidine-alkaline phosphatase conjugate. The presence of a hybridized probe was revealed using NBIT/BCIP color development. Details on the reverse hybridization are described in Stuyver et al. (1996), Stuyver et al. (1997) and Van Geyt et al. (1998).

### Statistical Analysis

277 children with a haematological malignancy or solid tumor and 205 C-diagnosed subjects were genotyped for the relevant SNPs in the different FCN genes.

The strength of association was reported as odds ratios (OR), indicating the factor by which the risk of developing a haematological malignancy or solid tumor in children is increased.

The 95% confidence interval (95% CI) is the interval computed from the sample data which, if the study repeated multiple times, would contain the true effect 95% of the time.

### Frequencies and Odds Ratios of FCN Genotypes in Children with a Haematological Malignancy or Solid Tumor and Control Individuals

The frequencies and odds ratios of the investigated SNPs in the FCN genes in children with a haematological malignancy or solid tumor and in healthy control individuals are indicated in Table 5.

The presence of the minor allele for the FCN1-rs1071583 polymorphism (exon9-Q275Q) has a protective effect on the risk of developing haematological malignancy or solid tumor in children [OR 0.71 (95% CI 0.55-0.93)].

TABLE 5
Allele frequencies and Odds Ratio's for SNPs
in FCN1 and FCN 2 gene - SNPs in children with
haematological malignancy or solid tumors

| | | FCN1 (Ex9)QQ |
|---|---|---|
| | | rs1071583 |
| | | WT: A-allele |
| SNP | n | Mut: G-allele |
| Controls | 172 | Total WT: 130 |
| | | Total Mut: 74 |
| pediatric | 227 | Total WT: 371 |
| oncology | | Total Mut: 183 |

| | |
|---|---|
| OR (95% CI) | 0.71 (0.55-0.93) |

EXAMPLE 3
Detection of nucleic acid variants in Ficolin genes from ICU patients
either without the development of sepsis, severe sepsis or septic shock
or with the development of this conditions.

### Patients Samples

A study was carried out based on blood samples from 170 patients (adults) admitted at the ICU. They were diagnosed with no sepsis (n=84) or sepsis (n=21), severe sepsis (n=15) or septic shock (n=50). For statistical calculations, the three ladder groups are sometimes taken together and called ‘total sepsis’ with n=86. For each blood sample, informed consent to participate in the study is available.

### Detection of Nucleic Acid Polymorphisms

To determine the presence or absence of nucleic acid variants in the FCN1 and FCN2 gene, the relevant 5′UTR, promotor and coding sequences (see Table 3) of the genes under study were amplified using biotinylated oligonucleotides. The polymorphisms were detected by use of a reverse hybridization method (Dot Probe Assay) with probes designed to recognize the polymorphisms as given in Table 3. After stringent washing at 56° C., hybridized probes were incubated with a streptavidine-alkaline phosphatase conjugate. The presence of a hybridized probe was revealed using NBIT/BCIP color development. Details on the reverse hybridization are described in Stuyver et al. (1996), Stuyver et al. (1997) and Van Geyt et al. (1998).

### Statistical Analysis

All the patients incorporated in the study were genotyped for 2 SNPs in FCN1 gene and 4 SNPs in the FCN2 gene.

The strength of association was reported as odds ratios (OR), indicating the factor by which the risk of developing sepsis is increased or decreased.

The 95% confidence interval (95% CI) is the interval computed from the sample data which, were the study repeated multiple times, would contain the true effect 95% of the time.

### Frequencies and Odds Ratios of FCN Genotypes in Patients Either without Sepsis or with the Development of Sepsis, or Severe Sepsis, or Septic Shock (Table 6)

a. Susceptibility to the Development of Sepsis

To this extend, allele frequencies for the 2 FCN genes were compared between the group of patients without sepsis versus the group who developed sepsis/severe sepsis or septic shock.
It was found that the presence of the minor allele (C-allele) for the FCN2-rs7865453 polymorphism and the minor allele (T allele) for the FCN2-rs7851696 polymorphism had a protective effect on the risk of developing sepsis, severe sepsis or septic shock [OR 0.57 (95% CI 0.29-1.12) and 0.46 (95% CI 0.23-094) respectively].
Splitting the generalized sepsis group into smaller subgroup representing a group developing sepsis, severe sepsis and septic shock allowed
   to define that the minor allele (A allele) for the FCN1-rs2989727 and the minor allele for the FCN1-rs10715832 polymorphism had a protective effect on the risk of developing severe sepsis [OR 0.42 (95% CI 0.16-1.08) and 48 (95% CI 0.18-1.18) respectively].
   To define that the to define that the minor allele (G-allele) for the FCN2-4A>G polymorphism and the minor allele (T allele) for the FCN2-T236M polymorphism resulted in a higher risk for the development of septic shock [OR 1.59 (95% CI 0.93-2.73) and 1.31 (95% CI 0.76-2.25) respectively].
      b. FCN SNPs as Prognostic Markers for the Development of Sepsis/Severe Sepsis or Septic Shock

To this extend, allele frequencies for the 2 FCN genes were compared between the different subgroups of patients who developed sepsis/severe sepsis or septic shock.
It was found that the presence that the minor allele (A allele) for the FCN1-rs2989727 and the minor allele for the FCN1-rs10715832 polymorphism had a prognostic use in predicting the development of septic shock if a patient is already affected with either sepsis or severe sepsis (see table 6)

TABLE 6
allele frequencies and Odds Ratio's for SNPs in FCN1 and FCN 2 gene -
SNPs in children with no sepsis, sepsis, severe sepsis or septic shock

| | | FCN1 (Prom) | FCN1 (Ex9)QQ | FCN2 (Prom) | FCN2 (Prom) | FCN2 (Ex8) | FCN2 (Ex8) |
|---|---|---|---|---|---|---|---|
| | | rs2989727 | rs1071583 | rs7865453 | -4AtoG | T236M | rs7851696 |
| | | WT: G-allele | WT: A-allele | WT: A-allele | WT: A-allele | WT: C-allele | WT: G-allele |
| SNP | n | Mut: A-allele | Mut: G-allele | Mut: C-allele | Mut: G-allele | Mut: T-allele | Mut: T-allele |
| no sepsis | 84 | Total WT: 62 | Total WT: 58 | Total WT: 142 | Total WT: 124 | Total WT: 122 | Total WT: 141 |
| | | Total Mut: 104 | Total Mut: 106 | Total Mut: 24 | Total Mut: 42 | Total Mut: 44 | Total Mut: 25 |
| sepsis total | 86 | Total WT: 58 | Total WT: 54 | Total WT: 157 | Total WT: 112 | Total WT: 114 | Total WT: 159 |
| | | Total Mut: 114 | Total Mut: 116 | Total Mut: 15 | Total Mut: 60 | Total Mut: 58 | Total Mut: 13 |

subgroups:

| | | | | | | | |
|---|---|---|---|---|---|---|---|
| sepsis | 21 | Total WT: 12 | Total WT: 14 | Total WT: 39 | Total WT: 28 | Total WT: 27 | Total WT: 39 |
| | | Total Mut: 30 | Total Mut: 28 | Total Mut: 3 | Total Mut: 14 | Total Mut: 15 | Total Mut: 3 |
| severe sepsis | 15 | Total WT: 6 | Total WT: 6 | Total WT: 28 | Total WT: 19 | Total WT: 19 | Total WT: 29 |
| | | Total Mut: 24 | Total Mut: 24 | Total Mut: 2 | Total Mut: 11 | Total Mut: 11 | Total Mut: 1 |
| septic shock | 50 | Total WT: 40 | Total WT: 34 | Total WT: 90 | Total WT: 65 | Total WT: 68 | Total WT: 91 |
| | | Total Mut: 60 | Total Mut: 64 | Total Mut: 10 | Total Mut: 35 | Total Mut: 32 | Total Mut: 9 |

Odds ratios (95% CI) susceptibility to develop sepsis

| | | | | | | |
|---|---|---|---|---|---|---|
| OR No sepsis vs | | | 0.57(0.29-1.12) | | | 0.46(0.23-0.94) |
| sepsis total | | | | | | |
| OR No sepsis vs | 0.42(0.16-1.08) | 0.48(0.18-1.18) | 0.42(0.09-1.89) | | | 0.19(0.03-1.49) |
| sepsis total | | | | | | |
| OR No sepsis vs | | | 0.66(0.3-1.44) | 1.59(0.93-2.73) | 1.31(0.76-2.25) | 0.56(0.25-1.25) |
| septic shock | | | | | | |

Odds ratios (95% CI) predicting severity of disease

| | | |
|---|---|---|
| OR Sepsis vs | 1.67(0.76-3.64) | |
| septic shock | | |
| OR Severe sepsis | 2.67(1.0-7.1) | 2.13(0.79-5.7) |
| vs septic shock | | |

EXAMPLE 4
Detection of nucleic acid variants in Ficolin genes from patients with
Rheumatoid arthritis and from control subjects

### Patients Samples

A pilot study comprising 203 RA patients has been extended with 139 patients making a total of 344 patients with confirmed Rheumatoid arthritis. The control group (C) consisted of 205 healthy individuals. From each blood sample, informed consent to participate in the study is available.

### Detection of Nucleic Acid Polymorphisms

To determine the presence or absence of nucleic acid variants in the FCN1, FCN2 and FCN3 gene, the relevant 5′UTR, promotor, IVS and coding sequences (see Table 3) of the genes under study were amplified using biotinylated oligonucleotides. The polymorphisms were detected by use of a reverse hybridization method (Dot Probe Assay) with probes designed to recognize the polymorphisms as given in Table 3. After stringent washing at 56° C., hybridized probes were incubated with a streptavidine-alkaline phosphatase conjugate. The presence of a hybridized probe was revealed using NBIT/BCIP color development. Details on the reverse hybridization are described in Stuyver et al. (1996), Stuyver et al. (1997) and Van Geyt et al. (1998).

### Statistical Analysis

344 patients with Rheumatoid arthritis and 172 C-diagnosed subjects were genotyped for 2 SNPs in FCN1 gene and 4 SNPs in the FCN2 gene. The strength of association was reported as odds ratios (OR), indicating the factor by which the risk of developing RA is increased.

The 95% confidence interval (95% CI) is the interval computed from the sample data which, were the study repeated multiple times, would contain the true effect 95% of the time.

### Frequencies and Odds Ratios of FCN Genotypes in Patients with Rheumatoid Arthritis (Table 7)

It was found that
1. the presence of the minor allele for the FCN1-rs2989727 and the minor allele of the FCN1-rs1071583 polymorphism had a protective effect on the risk of developing RA [OR 0.69 (95% CI 0.53-0.88) and OR 0.68 (0.53-0.88) respectively].
2. the presence of the minor allele (C-allele) for the FCN2-rs7865453 polymorphism and the minor allele (T allele) for the FCN2-rs7851696 polymorphism had a protective effect on the risk of developing RA [OR 0.61 (95% CI 0.42-0.88)] and 0.63 (95% CI 0.44-0.9) respectively].
3. the presence of the minor allele (G-allele) for the FCN2 A>G polymorphism and the minor allele (T allele) for the FCN2-T236M polymorphism resulted in a higher risk for the development of RA [OR 1.40 (95% CI 1.05-1.86)] and 1.52 (95% CI 1.15-20.2) respectively].

TABLE 7
allele frequencies and Odds Ratio's for SNPs in FCN1 and FCN 2 gene - SNPs in children with Rheumatoid arthritis

| | | FCN1 (Prom) | FCN1 (Ex9)QQ | FNC2 (Prom) | FCN2 (Prom) | FCN2 (Ex8) | FCN2 (Ex8) |
|---|---|---|---|---|---|---|---|
| | | rs2989727 | rs1071583 | rs7865453 | -4AtoG | T236M | rs7851696 |
| | | WT: G-allele | WT: A-allele | WT: A-allele | WT: A-allele | WT: C-allele | WT: G-allele |
| SNP | n | Mut: A-allele | Mut: G-allele | Mut: C-allele | Mut: G-allele | Mut: T-allele | Mut: T-allele |
| Controls | 205 | Total WT: 172 | Total WT: 168 | Total WT: 348 | Total WT: 318 | Total WT: 317 | Total WT: 344 |
| | | Total Mut: 238 | Total Mut: 242 | Total Mut: 62 | Total Mut: 92 | Total Mut: 93 | Total Mut: 64 |
| RA | 344 | Total WT: 228 | Total WT: 221 | Total WT: 621 | Total WT: 490 | Total WT: 477 | Total WT: 618 |
| | | Total Mut: 460 | Total Mut: 467 | Total Mut: 67 | Total Mut: 198 | Total Mut: 213 | Total Mut: 72 |

| | | | | | | |
|---|---|---|---|---|---|---|
| OR (95% CI) | 0.69 (0.53-0.88) | 0.68 (0.53-0.88) | 0.61 (0.42-0.88) | 1.40 (1.05-1.88) | 1.52 (1.15-2.02) | 0.63 (0.44-0.9) |

EXAMPLE 5
L-Ficolin levels in serum are, at least partly, determined by DNA
variations in the gene encoding FCN2 gene

To this extend, L-Ficolin levels were measured using a method published by Atkinson et al. 2004b. Briefly, antigen was captured using one monoclonal antibody (GN4) to L-ficolin on the solid phase and detected using another biotinylated monoclonal antibody (GN5). 55 samples with L-ficolin levels equal to or lower to 2.8 μg/mL [low L-ficolin levels] and 40 with levels of 4,5 μg/mL or higher [high L-ficolin levels] were used to evaluate a possible role of DNA polymorphisms in the FCN2 gene on the L-ficolin levels in serum. Chi square tests were used for statistical analysis

### Results (Table 8—FIG. 8)

the presence of the wild type allele (A-allele) for the FCN2-rs7865453 polymorphism and the wild type allele (G allele) for the FCN2-rs7851696 polymorphism is associated with high L-ficolin levels in serum (p>0.001)
the presence of the wild type allele (A-allele) for the FCN2-4A>G polymorphism and the wild type allele (C allele) for the FCN2-T236M polymorphism is associated with significantly reduced L-Ficoline levels in serum (p>0.001).

TABLE 8
Frequency of the wild type and mutant alleles for the four
FCN2 SNPs under study, in two distinct groups, one group
with high L-ficoline levels in serum (n = 40) and a
group with low L-ficolin levels in serum (n = 55)

| | high L-ficolin serum | | low L-ficoline serum | |
|---|---|---|---|---|
| | levels n = 40 | | levels n = 55 | |
| | WT alleles | Mut alleles | WT alleles | Mut alleles |
| FCN2 rs7865453 | 79 | 1 | 92 | 18 |
| FCN2 −4A > G | 32 | 38 | 88 | 36 |
| FCN2 T236M | 30 | 48 | 67 | 43 |
| FCN2 rs7851696 | 78 | 0 | 90 | 20 |

### REFERENCES

Atkinson A P M et al (2004a) L-ficolin in children with recurrent respiratory infections. Clin Exp Immunol, vol 138, issue 3, p 517
Atkinson A P, Cedzynski M, Szemraj J, St Swierzko A, Bak-Romaniszyn L, Banasik M, Zeman K, Matsushita M, Turner M L, Kilpatrick D C. L-ficolin in children with recurrent respiratory infections. Clin Exp Immunol. 2004b December; 138(3):517-20.
Ausubel F M, Brent R, Kingston R E, Moore D D, Seidman J G, Smith J A and Struhl K. Series Editor: Virginia Benson Chanda. Current Protocols in Molecular Biology 2003, John Wiley & Sons.
Celis J. E., Gromov P., Ostergaard M., Madsen P., Honore B., Dejgaard K., Olsen E., Vorum H., Kristensen D. B., Gromova I., Haunso A., Van Damme J., Puype M., Vandekerckhove J., Rasmussen H. H. (1996) Human 2-D PAGE databases for proteome analysis in health and disease: http://biobase.dk/cgi-bin/celis. FEBS Lett. 398: 129-134.
Christiansen O. B., Kilpatrick D. C., Souter V., Varming K., Thiel S., Jensenius J. C. (1999) Mannan-binding lectin deficiency is associated with unexplained recurrent miscarriage. Scand J Immunol. 49: 193-196.
Davidsson P., Westman A., Puchades M., Nilsson C. L., Blennow K. (1999) Characterization of Proteins from Human Cerebrospinal Fluid by a Combination of Preparative Two-Dimensional Liquid-Phase Electrophoresis and Matrix-Assisted Laser Desorption/Ionization Time-of-Flight Mass Spectrometry. Anal. Chem. 71: 642-647.
Den Dunnen J T and Antonarakis S E. Mutation nomenclature extensions and suggestions to describe complex mutations: A discussion. Hum. Mut. 2000; 15: 7-12.
Fujita T. Evolution of the lectin-complement pathway and its role in innate immunity Nat Rev Immunol. 2002 May; 2(5):346-53.
Ghindilis A. L., Pavlov A. R., Atanassov P. B. (eds.) (2002) Immunoassay Methods and Protocols. Humana Press, Totowa, N.J., US.
Gut I. G. (2001) Automation in genotyping of single nucleotide polymorphisms. Hum. Mutat. 17: 475-492.
Hummelshoj T et al. (2003) Abstracts/Molecular Immunology 40: 171-228
IFCC. (1987) Approved recommendation on the theory of reference values. Part. 5. Statistical treatment of collected reference values. Determination of reference limits. J. Clin. Chim Acta 1987; 170:S13-S32
Kilpatrick D. C. (2002) Mannan-binding lectin and its role in innate immunity. Transfusion Medicine 12: 335-351.
Kilpatrick D C, McLintock L A, Allan E K, Copland M, Fujita T, Jordanides N E, Koch C, Matsushita M, Shiraki H, Stewart K, Tsujimura M, Turner M L, Franklin I M, Holyoake T L. No strong relationship between mannan binding lectin or plasma ficolins and chemotherapy-related infections. Clin Exp Immunol. 2003 November; 134(2):279-84.
Klose J., Kobalz U. (1995) Two-dimensional electrophoresis of proteins: an updated protocol and implications for a functional analysis of the genome. Electrophoresis 16: 1034-1059.
Kuhlman M., Joiner K., Ezekowitz R. A. (1989) The human mannose-binding protein functions as an opsonin. J. Exp. Med. 169: 1733-1745.
Lu J, Teh C, Kishore U, Reid KB. Collectins and ficolins: sugar pattern recognition molecules of the mammalian innate immune system. Biochim Biophys Acta. 2002 Sep. 19; 1572(2-3):387-400.
Matsushita M, Endo Y, Fujita T. Cutting edge: complement-activating complex of ficolin and mannose-binding lectin-associated serine protease. J Immunol. 2000 Mar. 1; 164(5):2281-4.
Medzhitov R, Janeway C Jr. Innate immunity. N Engl J Med. 2000 Aug. 3; 343(5):338-44.
Miller M. E., Seals J., Kaye R., Levitsky L. C. (1968) A familial plasma associated defect of phagocytosis. Lancet 2: 60-63.
Minchinton R M, Liley H, Eisen D P. (2004) The body as fortress: innate immune surveillance. Vox Sang., 87 Suppl:30-4
Monroe et al. (1986) Amer. Clin. Prod. Rev. 5: 34-41
Nollau P, Wagener C. (1997) Methods for detection of point mutations: performance and quality assessment. IFCC Scientific Division, Committee on Molecular Biology Techniques. Clin Chem., 43(7): 1114-28.
O'Farrell P. H. (1975) High resolution two-dimensional electrophoresis of proteins. J Biol. Chem. 250: 4007-4021.
Patterson S. D., Aebersold R. (1995) Mass spectrometric approaches for the identification of gel-separated proteins. Electrophoresis 16: 1791-814.
Sambrook J., Fritsch E. and Maniatis T. (1989) Molecular cloning: a laboratory manual. Cold Spring Harbor Laboratory Press, Cold Spring Harbor, N.Y., USA.
Seelen M. A., Wieslander J. W., Sommarin Y., Persson A., Schlagwein N., Daha M. R., Roos A., the European Workgroup on Complement in Disease. Functional assays for the assessment of the activity of the three pathways of complement activation in a simple ELISA format. Mol. Immunol. (Special Issue: 9^{th} European Complement Workshop, 6-9^{th} September 2003, Trieste, Italy) Vol. 40: 194.
Stuyver L., Wyseur A., van Arnhem W., Hernandez F., Maertens G. (1996) A second generation line probe assay for hepatitis C virus. J. Clin. Microbiol. 34: 2259-2266.
Stuyver L., Wyseur A., Rombout A., Louwagie J., Scarcez T., Verhofstede C., Rimland D., Schinazi R. F., Rossau R. (1997) Line probe assay (LiPA) for the rapid detection of drug-selected mutations in the HIV-1 reverse transcriptase gene. Antimicrob. Agents Chemother. 41: 284-291.
Super M., Thiel S., Lu J., Levinsky R. J., Turner M. W. (1989) Association of low levels of mannan-binding protein with a common defect of opsonisation. Lancet 2: 1236-1239.
Syvanen A. C. (2001) Accessing genetic variation: genotyping single nucleotide polymorphisms. Nat. Rev. Genet. 2: 930-942.
Van Geyt C., De Gendt S., Rombaut A., Wyseur A., Maertens G., Rossau R., Stuyver L. (1998) A line probe assay for hepatitis B virus genotypes. In: R. F. Schinazi, J. P. Sommadossi, and H. Thomas (eds.). Therapies of viral hepatitis. International Medical Press, London, UK, pp. 139-145.
Wild D. (ed.) (2001) The Immunoassay Handbook 2^{nd} edition. Nature Pr., London, UK.
Yan J. X., Tonella L., Sanchez J. C., Wilkins M. R., Packer N. H., Gooley A. A., Hochstrasser D. F., Williams K. L. (1997) The Dictyostelium discoideum proteome—the SWISS-2DPAGE database of the multicellular aggregate (slug). Electrophoresis 18: 491-497.
Yokota Y., Arai T., Kawasaki T. (1995) Oligomeric structures required for complement activation of serum mannan-binding proteins. J. Biochem. 117: 414-419.

1. A method of identifying a subject at risk of or having, an indication associated with altered innate immunity, comprising detecting the presence or absence of at least one nucleic acid variant in at least one ficolin gene, or part thereof whereby the presence of at least one nucleic acid variant identifies whether a subject is at risk or has an indication associated with an altered innate immunity.

2. The method according to claim 1, whereby the presence or absence of the nucleic acid variant is detected in FCN1 gene, the FCN2 gene and/or the FCN3 gene.

3. The method according to claim 2, whereby the presence or absence of the nucleic acid variant is detected in:
the promotor region and/or in exon 9 of the FCN1 gene,
the promotor region, the 5′UTR region and/or exon 8 of the FCN2 gene, and/or
the IVS5 region of the FCN3 gene.

4. The method according to claim 3, whereby the nucleic acid variant is detected at:
position −1981 and/or position 7919 of the genomic DNA sequence of the FCN1 gene,
position −64, −4, 6424 and/or 6359 of the genomic DNA sequence of the FCN2 gene, and/or
position 3836 of the genomic DNA sequence of the FCN3 gene.

5. The method according to claim 1 wherein the nucleic acid variant is a substitution, deletion and/or insertion of at least one nucleotide.

6. The method according to claim 1 wherein the presence of at least one nucleic acid variant identifies a subject at risk of, or having, an increased or reduced susceptibility for disease.

7. The method according to claim 1 wherein the presence of at least one nucleic acid variant identifies a subject at risk of, or having, an increased severity of disease.

8. The method according to claim 1 wherein the presence of at least one nucleic acid variant identifies a subject at risk of, or having, a modified response to therapy for a disease.

9. The method according to claim 1 wherein the presence of at least one nucleic acid variant identifies a protective effect on the risk of developing a disease.

10. The method according to claim 1 wherein the presence of at least one nucleic acid variant identifies a subject at risk of, or having, an increased risk of transplant rejection.

11. The method according to claim 6 wherein the disease is an infection, an autoimmune disease, an inflammatory disease, cancer, cystic fibrosis, cardiovascular disease or Alzheimer's disease.

12. The method according to claim 11, wherein the infection is a bacterial, viral, fungal, parasitic or algae infection.

13. The method according to claim 12 wherein the infection is sepsis, severe sepsis, septic shock or otitis media.

14. The method according to claim 11, wherein the autoimmune disease is rheumatoid arthritis, spondyloarthropathy, systemic lupus erythematosus, multiple sclerosis, Crohn's disease, coeliac disease, Type I diabetes, Kawasaki disease, atopic disease, asthma, atopic dermatitis, dermatomyositis, Behçet's disease or Sjögren's Syndrome.

15. The method according to claim 11, wherein cancer is: (1) a solid tumor such as colon cancer, colorectal cancer, gastric cancer, cervical cancer, lung cancer, liver cancer, kidney cancer or brain cancer, or (2) a haematological malignancy selected from the group consisting of
a) Leukemias: acute myeloid leukemia (AML), acute lymphoid leukemia (ALL), chronic myeloid leukemia (CML) and chronic lymphoid leukemia (CLL),
b) Lymphomas: Hodgkins and non-Hodgkin's lymphomas and
c) Myelomas.

16. The method according to claim 11 wherein cardiovascular disease is bypass failure, atherosclerosis, myocardial reperfusion injury, coronary artery disease or heart disease.

17. The method according to claim 10 wherein the transplant is an organ or bone marrow.

18. The method according to claim 1, further comprising the detection of the presence or absence of a nucleic acid variant in any other gene.

19. The method according to claim 18 wherein the gene is selected from the group consisting of: BPI, CD14, beta-catenin, IL10, LBP, RP105, MBL2, MD-1, MD-2, MYD88, NOD1, NOD2, TLR1, TLR2, TLR3, TLR4, TLRS, TLR6, TLR7, TLR8, TLR9 and TLR10.

20. The method according to claim 1, wherein the detection for the presence or absence of the nucleic acid variant is performed by at least one of the following methods: hybridization, sequencing, PCR, primer extension, MLPA, OLA or restriction site analysis.

21. The method according to claim 1, wherein the detection of the presence or absence of the nucleic acid variant is determined in vitro in a biological sample obtained from said subject.

22. A method of identifying a subject at risk of, or having, an indication associated with altered innate immunity, comprising measuring the concentration or functionality of at least one ficolin protein in a biological sample, wherein an increased or decreased ficolin concentration or altered functionality compared to a reference value representing a known health status indicates that said subject is at risk or suffers from a disorder associated with altered innate immunity.

23. A kit for identifying a subject at risk of, or having, an indication associated with altered innate immunity, comprising:
(a) a means for detecting the presence or absence of one or more nucleic acid variants in a ficolin gene; and
(b) a means for determining, from the nucleic acid variant detected with the means of step (a), whether the subject is at risk of, or has, an indication associated with altered innate immunity.

24. A kit for identifying a subject at risk of, or having, an indication associated with altered innate immunity, comprising:
(a) an antibody for detecting the presence or absence of a ficolin protein;
(b) a means for determining, from the ficolin protein detected with the means of step (a), whether the subject is at risk of, or has, an indication associated with altered innate immunity.

25. A method for selecting an appropriate treatment or therapeutic agent for a subject at risk of or having, an indication associated with altered innate immunity, comprising
determining the risk or the presence of an indication associated with altered innate immunity by the method according to claim 1, and
selecting an appropriate treatment or therapeutic agent which elicits a high responsiveness and/or low adverse effects in said subject.

26. The method according to claim 2 wherein the nucleic acid sequence of the wild type FCN1 gene is identified by SEQ ID NO: 11, the nucleic acid sequence of the wild type FCN2 gene is identified by SEQ ID NO:12 and the nucleic acid sequence of the wild type FCN3 gene is identified by SEQ ID NO: 13, or the complementary thereof.

27. The method according to claim 26 wherein the nucleic acid variant is detected in the corresponding cDNA or RNA sequence.

28. A method for detecting the presence or absence of a nucleic acid variant in the FCN2 gene comprising determining in a sample the nucleotide at position −4 and/or 6359 in the FCN2 gene consisting of a wild-type sequence as defined by NT_{—}0019501, or the complementary thereof, and thereby detecting the presence or absence of the nucleic acid variant.

29. The method according to claim 28 whereby the detection of the presence or the absence of the nucleic acid variant is performed by at least one of the following methods: hybridization, sequencing, PCR, primer extension, MLPA, OLA or restriction site analysis.

30. The method according to claim 28 comprising the amplification of the region comprising position −4 and/or 6359 using at least one pair of primers specifically hybridizing to the sequence as represented by NT_{—}019501, or the complementary thereof.

31. A isolated nucleic acid comprising at least 8 nucleotides specifically hybridizing with the nucleic acid sequence of the wild type or variant sequence of the FCN2 gene including position −4 and/or 6359, or the complementary thereof.

32. The isolated nucleic acid according to claim 31 for use as a primer or probe.

33. A kit for detecting the presence or absence of a nucleic acid variant at position −4 and/or 6359 in the FCN2 gene comprising:
at least one oligonucleotide suitable for detection of a target ficolin polynucleic acid and/or at least one oligonucleotide pair suitable for amplification of a target ficolin polynucleic acid;
when appropriate, an agent for denaturing nucleic acids;
when appropriate, an enzyme capable of modifying a double stranded or single stranded nucleic acid molecule;
when appropriate, a hybridization buffer, or components necessary for producing said buffer;
when appropriate, a wash solution, or components necessary for producing said solution;
when appropriate, a means for detecting partially or completely denatured polynucleic acids and/or a means for detecting hybrids formed in the preceding hybridization and/or a means for detecting enzymatic modifications of nucleic acids; and
when appropriate, a means for attaching an oligonucleotide to a known location on a solid support.

34. An isolated oligonucleotide consisting of 10 to 30 nucleotides for detecting the presence of one or more nucleic acid variants in SEQ ID NO: 11, 12 or 13, or the complementary strand.

35. The oligonucleotide of claim 34, whereby the nucleic acid variant is located at position −1981 or 7919 of SEQ ID NO: 11, position −64, −4, 6424 or 6359 of SEQ ID NO:12, or position 3836 of SEQ ID NO:13.

36. A pair of primers suitable for amplification of a target FCN polynucleic acid.

37. The pair of primers according to claim 36 whereby the target polynucleic acid is the promotor region or exon 9 of the FCN1 gene, the promotor region, the 5′UTR region or exon 8 of the FCN2 and/or NS5 of the FCN3 gene, or part thereof.

38. The pair of primers according to claim 37 whereby the target polynucleic acid or part thereof comprises position −1981 and/or 7919 of SEQ ID NO: 11, position 64, −4, 6424 and/or 6359 of SEQ ID NO: 12, or position 3836 of SEQ ID NO: 13.

39. The kit according to claim 23 wherein the nucleic acid sequence of the wild type FCN1 gene is identified by SEQ ID NO: 11, the nucleic acid sequence of the wild type FCN2 gene is identified by SEQ ID NO:12 and the nucleic acid sequence of the wild type FCN3 gene is identified by SEQ ID NO: 13, or the complementary thereof.
